(19) United States
(12) Patent Application Publication
(10) Pub. No.: US 2018/0211724 A1
(43) Pub. Date: Jul. 26, 2018

(54) SYSTEM AND METHOD FOR HEALTHCARE BILLING VERIFICATION

(71) Applicant: Kevin Sunlin Wang, Flushing, NY (US)

(72) Inventor: Kevin Sunlin Wang, Flushing, NY (US)

(21) Appl. No.: 15/934,677

(22) Filed: Mar. 23, 2018

Related U.S. Application Data
(63) Continuation-in-part of application No. 15/474,685, filed on Mar. 30, 2017.
(60) Provisional application No. 62/479,065, filed on Mar. 30, 2017.
(60) Provisional application No. 62/421,086, filed on Nov. 11, 2016.

(51) Int. Cl. G16H 40/20

(52) U.S. Cl. CPC G16H 40/20; G06F 17/30241

(57) ABSTRACT

The invention relates generally to health care management methods and systems, and more particularly, to a home care logistics and quality assurance tracking system and method that facilitates accountability of caregivers visiting, caring for, and treating patients. Upon receipt of a service request for a patient, the system establishes a field of acceptability for compliance. The field of acceptability includes a service location, a plurality of locations distinct from the service location, a service time, and a time period that includes the service time. The system identifies compliance or non-compliance with the service request by comparing the field of acceptability with location data and time data from the caregiver's and patient's computing devices, which may also be utilized to establish new temporary field boundaries. Upon identifying compliance, the system can automatically adjust a billing request to match a service request and authorize payment.

### CROSS-REFERENCE TO RELATED APPLICATIONS

The present application is based on and claims priority to U.S. Provisional Pat. Appl. Ser. No. 62/479,065, filed on Mar. 30, 2017, and is a continuation-in-part of U.S. patent application Ser. No. 15/474,685, filed on Mar. 30, 2017, which is based on and claims priority to U.S. Provisional Pat. Appl. Ser. No. 62/421,086, filed on Nov. 11, 2016, the disclosures of which are all incorporated by reference herein in their entireties.

### FIELD OF THE INVENTION

This invention relates generally to health care management systems, and more particularly, to a home care logistics and quality assurance tracking system and method that facilitates accountability of caregivers visiting, caring for, and treating patients. The system verifies caregiver compliance with a service request by comparing location data and time data from caregiver and patient computing devices with a dynamically updated field of acceptability.

### BACKGROUND

As the senior citizen population in the United States increases, so too do home health care costs. Given a choice between an extended hospital stay, frequent visits to a doctor, or being taken care of at home, most patients prefer home care. Various home health care agencies facilitate home healthcare services and are often paid by one or both of federal and/or state programs such as Medicaid and Medicare, or private health insurance. The home health care agencies dispatch caregivers to patient homes (e.g., nurses and aides) who have expertise in different healthcare areas with varying levels of proficiency. The caregivers are dispatched in accordance with a clinical plan of care instructed and/or provided by the patient's primary care physician(s), which may include a particular number of visits per week and a recommended length of each visit depending on the patient's needs. Adherence to the clinical plan of care is important in obtaining a positive outcome and improving the patient's health. Additionally, a patient's condition and well-being are often heavily influenced by the attendance and level of care provided by each visiting caregiver, as well as their compliance with the clinical plan of care.

A health care agency administrator is generally responsible for handling all visit information, including that which is used for billing and payroll. However, the more patients an agency accepts, the more difficult managing visits and billing information becomes. Thus, billing errors and fraudulent time-keeping are often missed and represent some of the primary preventable costs in the home health care field. In order to minimize these problems, Administrators often must delegate service visit information for coordinators to handle.

Federal regulations require a caregiver to self-report employment times through an Electronic Visit Verification (EVV). The industry standard for EVV typically requires a caregiver arriving at a patient's premises to call the home health agency that employs him/her to enable the agency to track a caregiver's arrival and departure times. One problem with EVV is that it fails to utilize various modern technological tools employed through mobile computing devices such as smartphones and tablets, and even wearable technology, into the verification process.

Proper tracking of caregiver work time is an important part of visit verification. Some caregivers work for more than one home health care agency and may visit different patients within the same day or the same week. Such workloads render the caregiver's services more difficult to properly track and report, as different agencies may utilize different service codes and procedures. A caregiver who tends to multiple patients before entering any time-related information may, for example, inadvertently schedule overlap between different agencies which employ the caregiver. Currently, many home health care systems rely heavily on the visiting caregivers' self-reporting and have a very difficult time monitoring their caregivers due to lack of direct supervision. Caregivers sometimes attempt to defraud the agencies and the patients by reporting services that were either not performed or poorly performed within the time window. Caregivers sometimes even have their own family members (who may be patients) join in on these fraudulent practices. Without oversight, it is difficult to judge whether the caregiver actually completed any of the work that was reported back to the agency. The only way for an agency to become aware of and address this issue is through patient complaints, visiting the locations, and relying on phone calls to check in with the patient. These methods are both time consuming and inconvenient. Disadvantages and problems can also arise for the caregiver with respect to an unreasonable patient. For example, when some patients are unhappy with a caregiver's level of service, even though the level of service provided may have been adequate, the patients will report it as improper. Without evidence of the service provided during the visit, agencies are sometimes forced into agreeing with the patient. Since self-reporting is not governed by an agency's supervision, an abundance of miscommunication, fraud, and/or abuse may arise by certain caregivers and/or certain patients.

In an attempt to overcome these time issues where an EVV time does not match the caregiver's originally scheduled time, the only way for time reconciliation is through a physical timesheet given to the coordinators or administrator. Timesheets are often used as means of tracking services provided. These timesheets allow for entry of the services performed by the caregiver, date of service, caregiver identifying information, patient information, and a patient signature confirming that such services were provided. The patient signs the timesheet after all services have been performed by the caregiver. These timesheets, however, burden the home health agency with extra paperwork, in addition to handling all of the other agency functions. Furthermore, storing the timesheets becomes an extra burden on the entire system as these paper timesheets are very time-consuming and costly to maintain. Moreover, such practices are no more accurate and are just as susceptible to fraud. Forgery seems to be common, and if the patient has a good relationship with the caregiver, the patients may dishonestly sign off for them, which allows the caregiver to receive compensation from the agency for little or no work.

Another technological drawback is the lack of location tracking for the caregiver and/or patient. Once the caregiver is clocked in through EVV, it is very difficult to track whether the caregiver is actually providing service at the patient's location. Although the EVV log may show that the caregiver has clocked in, the patient may not even know the whereabouts of the caregiver. Three situations typically occur. First, the caregiver may actually be on-site providing the scheduled service to the patient. Second, the caregiver may have never arrived at the patient's location to provide service, and instead clocked in through EVV from another location. Third, the caregiver may be on a shift with one agency and calling to clock in with another agency simultaneously, thus committing fraud through multiple instances of billing for two or more patients where no care was provided. Fourth, multiple caregivers may collude to claim being on shift for multiple patients at the same time. Fifth, two caregivers may claim to be on a shift for the same patient, thus committing fraud through instances of billing for the same services provided to one patient where only one caregiver actually provided services.

One type of tracking system known in the art is the United State's Global Positioning System (GPS), which employs a satellite-based geolocation system in which a portable device may acquire signals from a GPS satellite constellation and triangulate its position. Other tracking systems known in the art include China's BeiDou network, Russia's GLONASS service, India's Regional Navigation Satellite System (IRNSS) having the operational name NAVIC, Japan's Quasi-Zenith Satellite System (QZSS), and Europe's Galileo network. The wide-spread availability of these tracking services, especially GPS technology, has led to a wide range of uses. These devices, however, are prone to manipulation, particularly if the employee does not carry it. While an employer may install one of these devices in a work vehicle, the employee can simply park the vehicle at a designated patient location but go elsewhere for personal reasons. Additionally, since these devices may not be tamper-proof, an employee can tinker with the devices so they do not transmit information properly.

Existing electronic billing verification systems are either too strict, too broad, or not updated frequently enough to allow for home healthcare services billing acceptance. While home healthcare billing has largely transitioned to electronic billing, the field still faces unique obstacles, and technical advances have largely ignored the problems associated therewith. Technology-based verification features as disclosed herein provide a technological solution to verify that a home healthcare service was indeed provided, or that any deviation in time or location was due to a reasonable or legitimate cause, verified by establishing a “field of acceptability.” The field of acceptability provides a balance between too-strict systems and too-permissive systems.

### SUMMARY OF THE INVENTION

This summary is not intended to identify or point to essential features or limit the scope of the subject matter claimed herein. The following description includes a computer-implemented system and method for establishing and updating a field of acceptability for verifying a billing request for caregiver services (e.g., services of a home healthcare provider such as an aide, a therapist, etc.). The system may implement at least one or more servers, one or more geographical information systems (GIS), and one or more databases, which are arranged in a network connecting to a caregiver module. The caregiver module may include at least a geolocation identifier, a geo-aware camera (or a camera having location awareness), and a clock mechanism to identify a current time and date. The system includes a non-transitory computer-readable storage medium that stores instructions to programmatically carry out tasks. The tasks, which include receiving a billing request and its related data, can be deployed by one or more servers.

An exemplary embodiment of the inventive disclosure provides a device, system, and method for providing adjustable geolocation and time for caregiver visit verification at a patient's home or other location(s). A geolocation identifier identifies the location of a device. A clock mechanism obtains a time stamp. A processor generates a request for billing and transmits it through a means of communication to a central system which formats the billing request and determines whether it is acceptable based on predetermined rules. A user engagement panel is provided for a caregiver to provide reasons for acceptance of the billing request if the billing request is not accepted, according to predefined rules. Verification through the user engagement panel may reduce fraud by ensuring that the caregiver provides sufficient care in relation to his/her assigned tasks, is compliant with the attendance requirements of the home healthcare agency employing him/her, is within the scope of any insurance company requirements, has not left the patient's vicinity for any reason outside of the scope of employment, and/or has not engaged in fraudulent billing activity, such as substituting an unauthorized caregiver for himself/herself.

The field of acceptability is established by the system for healthcare service verification, where predetermined rules govern the parameters of it. At relevant points, a determination that a healthcare caregiver or provider is within or not within the field of acceptability may be used to start the process of determining whether the caregiver is eligible for payment for services provided in a billing request. If the caregiver is within the field of acceptability, then the generated billing request may be automatically adjusted to match certain of the designated information received to allow acceptance of the billing request by the system. If the caregiver is not within the field of acceptability, then he/she may be prompted to submit one or more reasons and/or a photograph enriched with geolocation and time metadata. This information may be used as evidence of legitimate reasons for failing to be within the field of acceptability and may be submitted via a user engagement panel. The user engagement panel provides a user interface that includes or is operatively associated with a platform to collect one or more ratings from one or more additional users to participate in rating, and by extension, verifying, the information the caregiver submits. A user with firsthand experience can review the information, including the one or more reasons provided by the caregiver, and confirm or deny that the caregiver's reasons were legitimate.

An exemplary embodiment of the inventive disclosure provides a computer-implemented method, comprising receiving a service request for a patient, the service request including a service location and a service time, establishing a field of acceptability for complying with the service request in accordance with one or more predetermined rules, periodically receiving location data and time data generated by a first computing device associated with a caregiver assigned to service the service request, receiving a billing request associated with the service request, automatically identifying, without user input, compliance with the service request by comparing the location data and the time data generated by the first computing device with the field of acceptability in accordance with the one or more predetermined rules, and responsive to identifying compliance with the service request, automatically adjusting at least a portion of the location data generated by the first computing device to match the service location of the service request.

Another exemplary embodiment of a computer-implemented method of the inventive disclosure comprises receiving a service request for a patient, the service request including a service location and a service time, establishing a field of acceptability in accordance with one or more predetermined rules for complying with the service request, periodically receiving location data and time data generated by a first computing device associated with a caregiver assigned to service the service request, automatically identifying, without user input, non-compliance with the service request by comparing the location data and the time data generated by the first computing device with the field of acceptability in accordance with the one or more predetermined rules, and responsive to identifying the non-compliance with the service request, transmitting a notification to the caregiver of non-compliance with the service request, and at least one of prompting the caregiver to comply with the service request or prompting the caregiver to submit at least one of a reason or evidence of a reason for the non-compliance with the service request.

An exemplary embodiment of a computer-implemented system for verifying compliance with a home healthcare service comprises a database for storing a plurality of service requests and first location data associated with a plurality of a patients, each service request including one or more requested services and an anticipated duration of the requested service, a server configured to receive second location data generated by one or more location identifier units of computing devices associated with caregivers, wherein the second location data identifies locations of the computing devices determined using the location identifier units at times the caregivers are physically at the locations, and one or more processors programmed to: receive a service request for a patient, the service request including a service location and a service time, establish a field of acceptability in accordance with one or more predetermined rules for complying with the service request, periodically receive location data and time data generated by a first computing device associated with a caregiver assigned to service the service request, receive a billing request associated with the service request, automatically identify, without user input, compliance with the service request by comparing the service location and the service time, respectively, with the location data and the time data generated by the first computing device in accordance with the one or more predetermined rules, and responsive to identifying compliance with the service request, automatically adjust at least a portion of the location data or the time data generated by the first computing device to match the service location or the service time of the service request.

Alternatively, the method may comprise establishing a set of service rules for defining the field of acceptability, the set of service rules including a predefined region for the service location, one or more locations not within the predefined region for the service location, the service time, and a time period which includes the service time and responsive to identifying compliance with the service request, modifying the set of service rules to include the location data generated by the first computing device in defining the field.

The inventive disclosure relates generally to health care management systems, and more particularly to a home care logistics and quality assurance tracking systems and methods that facilitate accountability of caregivers visiting, caring for, and treating patients with at least the following objectives:

To provide a customizable billing verification method and system for home health care services based on a predetermined set of rules for establishing a field of acceptability for defining an acceptable area for performing such services.

To establish a preliminary field of acceptability for a service request that is later converted to a permanent field of acceptability based on receipt of additional time and location data from the caregiver assigned to the service request and/or from additional users.

To establish a temporary addition to the field of acceptability by defining an additional area/region or location whose boundaries are defined based on where the caregiver provides services (e.g., by tracking a pair of computing devices associated with the patient and caregiver outside of the permanent field), and upon receipt of approval by the patient, the caregiver, the agency, and/or the insurer, modifying the field of acceptability to include the temporary addition.

To provide a customizable billing verification method and system for home health care services, whereby a caregiver is periodically monitored to verify compliance with a field of acceptability for providing the services based on a predetermined set of rules.

To provide a system configured to dynamically update the field of acceptability congruent with changes in patient and caregiver predetermined rules.

To enable patients to customize and dynamically update predetermined rules governing the field of acceptability for particular service requests.

To provide various sets of indicators to facilitate compliance with the established field of acceptability for a particular service request.

To provide a special purpose device that may be used for tracking home health care patients and caregivers to enable verification of billing information associated with the caregivers providing service to the patients.

To enable verification of billing information associated with caregivers providing services to patients by automatically identifying, without user input, compliance with a service request by comparing the field of acceptability with location data and time data generated by a first computing device in accordance with predetermined rules, and, responsive to identifying compliance with the service request, automatically adjusting the location data or the time data generated by the first computing device to match the service location or the service time of the service request.

To update an acceptable service field (field of acceptability) based on aggregate data from one or more caregivers servicing a plurality of service requests for a patient.

To automatically establish a temporary or permanent field of acceptability for a new route between two approved service locations based on caregiver travel time along the route.

To reduce the number of unnecessary non-compliant alerts to caregivers by dynamically updating the field of acceptability.

To establish a field of acceptability for service between and around multiple approved locations with particularity by tailoring it to specific pathways along routes which caregivers travel, and by not including alternative geographic regions which have not been navigated or time tested by caregivers.

To facilitate connectivity between a coordinator and caregivers by displaying changes to a service request in a dispatch web portal for the coordinator and in a caregiver's device associated with a caregiver assigned to the service request, and dynamically updating and storing the changes in a database without any phone call communication.

Other objects, features, and characteristics of the inventive disclosure, as well as the methods of operation and functions of the related structural elements, and the combination of parts and economies of development and manufacture, will become more apparent upon consideration of the detailed description below with reference to the accompanying drawings, all of which form a part of this specification.

### BRIEF DESCRIPTION OF THE DRAWINGS

A further understanding of the inventive disclosure can be obtained by reference to a preferred embodiment set forth in the illustrations of the accompanying drawings. The drawings are not intended to limit the scope of this invention, which is set forth with particularity in the claims as appended or as subsequently amended, but merely to clarify and exemplify the inventive disclosure. Accordingly, a more complete appreciation of the inventive disclosure and many of the attendant aspects thereof may be readily obtained as the same becomes better understood by reference to the following detailed description when considered in conjunction with the accompanying drawings, where:

FIG. 1 is a schematic diagram of an exemplary caregiver billing verification system, including a computing system and one or more peripheral computing devices for use in accordance with exemplary embodiments of the inventive disclosure;

FIG. 2 is a diagram illustrating an exemplary structure of data stored in a database, and how organization of the data may be carried out in accordance with exemplary embodiments of the inventive disclosure;

FIG. 3 is a diagram of an exemplary computing device and associated components in accordance with exemplary embodiments of the inventive disclosure;

FIG. 4 is a schematic diagram further illustrating exemplary system components for home healthcare electronic billing verification and caregiver tracking in accordance with exemplary embodiments of the inventive disclosure;

FIG. 5A is a flowchart illustrating an exemplary workflow for making a billing request adjustment for a provision of service in accordance with an exemplary embodiment of the inventive disclosure;

FIG. 5B is a flowchart illustrating an approach for adjusting a billing request or information identified therein with regard to a geolocation that is not the designated location but deemed to be within a field of acceptability in accordance with an exemplary embodiment of the inventive disclosure;

FIG. 5C is a flowchart illustrating an approach for adjusting a billing request or information identified therein with regard to a time that is not the designated time but deemed to be within the field of acceptability in accordance with an exemplary embodiment of the inventive disclosure;

FIG. 6A is a flowchart illustrating an approach for establishing and updating the field of acceptability in accordance with an exemplary embodiment of the inventive disclosure;

FIG. 6B is a flowchart illustrating an approach for establishing a temporary field of acceptability in certain circumstances in accordance with an exemplary embodiment of the inventive disclosure;

FIG. 6C is a flowchart illustrating an approach for updating the temporary field of acceptability in accordance with an exemplary embodiment of the inventive disclosure;

FIG. 7 is a diagram illustrating the application of the field of acceptability based on distance in accordance with an exemplary embodiment of the inventive disclosure; and

FIG. 8 is a flowchart illustrating an alternative approach for creating a temporary field of acceptability and updating the permanent field of acceptability in accordance with exemplary embodiments of the inventive disclosure.

### DETAILED DESCRIPTION

Detailed illustrative embodiments of the inventive disclosure are disclosed herein. Specific exemplary embodiments that may be practiced are shown by way of illustration and explanation. The present disclosure is not intended to be limited to the specific terminology selected, and it will be understood that each specific element includes all technical equivalents which operate in a similar manner. However, techniques, methods, systems, and operating structures in accordance with the inventive disclosure may be embodied in a wide variety of forms and modes, some of which may be quite different from those in the disclosed embodiment. Consequently, the specific structural, functional and step-by-step details disclosed herein are merely representative, yet in that regard, are deemed to afford the best embodiment for purposes of disclosure, and to provide a basis for the claims herein which define the scope of the inventive disclosure. The embodiments herein are described in sufficient detail to enable those skilled in the art to practice the embodiments, and it is to be understood that logical, mechanical, and other changes may be made without departing from the scope of the embodiments. The following detailed description is therefore not to be taken in a limiting sense.

It will also be appreciated that various modules of the systems and methods described herein may be implemented in part by using an interfacing mobile application (app) on an internet-enabled mobile device's operating system, such as, for example, Android, iOS, or Windows OS, and in part by using a web portal interface, and that different types of users may utilize different functionalities of the system. Such users or subscribers can include, for example, one or more caregiver(s) or patients. Patient(s) as used herein can include anyone, including, for example, one or more individuals, entities, or one or more individuals from an entity, who request services for home healthcare, and may alternatively referred to as client or customer. A “patient” can refer to anyone who registers with the system, either an individual or individuals from an entity, who request services for home healthcare, regardless of the type of services (e.g., transport services, caregiver services, or any combination of services thereof). These patients are primarily senior citizens who require constant supervision by a certified caregiver. The care provided may be home health care or another relevant type of care, whether medical or nonmedical. Conversely, “caregiver” is used herein as a term referring to an individual who looks after, cares for, or provides service(s) for a patient, including paid or unpaid work, which may or may not be in a medical capacity. The term “caregiver” is also intended to encompass visiting medical professionals, such as home health aides, nurses, doctors, other health care professionals, a patient's family member(s), friend(s) or assistant(s) who is/are certified caregiver(s), etc. As patients and service providers alike may use the methods and systems described herein, both may generally be referred to as a “user” or “users,” in addition to being referred to by specific user type corresponding to the role they play with respect to the service request.

“Care” as used herein encompasses all tasks each caregiver is specified to perform on patient visits. These tasks include but are not limited to: household chores at a patient's home, assisting the patient with showering and/or bathing, preparing breakfast, lunch, dinner, and/or snacks, ambulation, accompanying the patient to medical visits, medicine reminders, vital checks, running errands, and purchasing groceries. In addition, “home health care agency” as used herein can refer to an entity which coordinates caregiver visits and accepts or denies potential patients. Home health care agency coordination may include booking a caregiver to a certain patient, paying the caregiver for services rendered, maintaining records, billing etc. “administrator” as used herein can apply to a person who manages all scheduling, employment, and patient intake, creates user roles, obtains patient information and permission, delegates different tasks to different user roles, and bills tasks within a home health care agency. “coordinator” as used herein can refer to an individual within the agency who handles all scheduling related tasks between caregivers and patients and ensures that all calendar functions are met. “Vendor(s)” as used herein can refer to a broker or other business entity, government office, or individual who brokers a service on behalf of a patient. Additionally, “geo-fencing” as used herein can refer to a perimeter or boundary surrounding a geographic area, represented as a square, circle, or any other shape or region.

A request for any type of healthcare service is generally referred to herein as a “service request” or “service visit(s)” throughout the disclosure, though other terms may be utilized. “Services” herein may refer to caregiver services within and around a patient's home, residence, and/or other service locations. In certain instances, “services” herein may take place in hospitals or other medical facilities. Furthermore, “customized” as used herein may modify the nature of the services and can refer to the preferences of a patient or the preferences or limitations of a caregiver rendering the services. Regardless of the type of service, a “service provider” as used herein may be a single individual such as a caregiver, a group of people, or an affiliate of a private business entity such as a home healthcare agency which dispatches caregivers to provide services at a patient's home, transport services or both. The service provider's ability to provide services depends on what tools he/she/they has/have readily available. For example, in the case of accompanying a patient somewhere, a vehicle may be necessary, which a caregiver may use if he/she drives to the patient's home instead of using public transportation.

According to an exemplary embodiment of the inventive disclosure, a method, system, and special purpose device may be used for tracking home health care patients, caregivers, and devices associated therewith. Tracking may enable creating or verifying billing information associated with services rendered to a patient by a caregiver. A caregiver often does not arrive on time or at a correct location. In such situations, the caregiver's failure to arrive on time or at the correct location occurs despite a good faith attempt. The caregiver may have had the wrong address. In other situations, the caregiver might have simply failed to show up because he/she decided not to work. In yet other situations, the caregiver and the patient may collude by coordinating visit verifications or clock-in times. These problems may be addressed at least in part by incorporating technology-based time and location verification features.

Herein, the example of home health care is used to show how an exemplary embodiment of the inventive disclosure may be implemented. However, the use of this example is not intended to limit the scope of what is claimed. It is to be understood by one having ordinary skill in the art that these concepts may be readily applied to other industries and examples where work may be done remotely and reported back to a central office, such as plumbing, electrical work, utilities repair and installation, etc. Essentially, it is to be understood that there may be a great deal of other relevant work situations outside of home health care where any one or a combination of concepts described herein may be applied.

As disclosed, a device, system, and method are directed to tracking a worker or caregiver directly in comparison to tracking a customer or patient. The geolocations of the caregiver and the patient may be tracked unless prohibited by any relevant laws. The geolocation of the caregiver may be dynamically compared to the geolocation of the patient. Predetermined rules may define the patient's location, other acceptable locations for a caregiver to provide services to the patient, a reasonable distance as to how far the caregiver is allowed to be from the patient, the services that the caregiver is to provide pursuant to the service request, etc. In the home healthcare industry, the predetermined rules are typically set or established by an insurance company, healthcare provider, or other entity responsible for paying for the healthcare services. Predetermined rules may additionally or alternatively be set by one or more agencies employing caregivers. Timing information may also be tracked, and may include a start time, an end time, and a duration of the service provided. A field of acceptability may be established and compared to the tracked geolocation information and/or timing information. A field of acceptability for location and a field of acceptability for time may be utilized individually or in combination. Furthermore, the predetermined rules may require the worker to “check in” at certain intervals by providing verification information such as location, identity verification, or both, in conjunction with the patient. As used herein, the term or phrase “field of acceptability” may also be referred to as “service field.”

Caregivers may periodically be provided with a notification to inform them of non-compliance with the service request and/or predetermined rules. If the caregiver is determined to be within the field of acceptability by the system, then the caregiver may successfully provide care and the services may be properly billed by the home healthcare agency, or in the case of a private caregiver, all services performed will be properly compensated while the caregiver is within the field of acceptability. However, if the caregiver is not within the field of acceptability (e.g., not on time, not at or within a region associated with a correct location, a new location not recognized, etc.), then the caregiver may be notified and/or prompted to provide an explanation for not being within the field. If the caregiver provides at least one reason, then the reason(s) provided may need to be verified by the agency. The system may store a set of default reasons based on common reasons various caregivers state or provide a fill-in field on a user interface of a user engagement panel for caregivers to provide a more detailed explanation. After review, the administrator may accept or reject the reasons provided.

Additionally, a caregiver may take a metadata enriched photograph as partial evidence to supplement at least one of the reasons provided. The metadata may include a geolocation identified by a GPS in the caregiver and/or patient's device, as well as a time when the photograph was taken, which can be timestamped by an internal clock mechanism. Accordingly, a determination that the caregiver is within the field of acceptability may be used in the process of determining whether the caregiver is eligible for payment for services rendered, as well as whether the home healthcare agency may bill insurance companies for the services that the caregiver provided. Field of acceptability-based adjustments are based on compliance with predetermined rules for timeframe(s) and/or distance(s) and/or patient location(s). The adjustment(s) may be made manually or automatically by the system.

A caregiver or patient interval check-in prompt may also be employed for purposes of time verification. At certain intervals during each service visit, which may vary from each location, caregiver, or patient, the caregiver or patient may be notified that he/she has to perform a check-in with their respective device. A check-in may ask for a fingerprint or other type of identification, or an explanation of what service the caregiver may currently be performing or has performed. This type of verification may be used to reduce fraud by ensuring that not only is the caregiver providing sufficient care in relation to his/her assigned tasks, but also compliant with the attendance requirements of the home healthcare agency employing him/her, has not left the patient's vicinity for any reason outside of the scope of employment, and has not engaged in fraudulent billing activity, such as substituting an unauthorized and unscheduled caregiver.

It is to be understood herein that the methods and systems described may be implemented in at least hardware, software, or firmware, and may employ specifically configured processors or special purpose processing means. Such methods and systems may utilize software-implemented instructions stored on one or more devices that are tangibly embodied, such as a semiconductor memory device (e.g., RAM, ROM, PROM, EEPROM, or Flash-Programmable RAM), a magnetic memory device (e.g., a diskette or fixed hard disk), an optical memory device (e.g., CD-ROM or DVD), a PC card (e.g., PCMCIA card), or other memory device. Those instructions may be implemented by any suitable device with suitable architecture. Further, it will be appreciated by one of ordinary skill in the art that since these systems may be implemented in software, the constituent system components may differ in certain exemplary embodiments in response to how an application is programmed.

The methods and systems disclosed herein may be in part embodied in one or more servers. Each server or servers may be employed for one or more specific functions. A database server may be used to deploy one or more databases and optimize database queries. A server may be configured to act as the network server, and connection to one or more peripheral devices (e.g., a mobile device or one or more remote devices such as a computer or caregiver to patient synced device(s) at another location) may be established. Such connections may be accomplished through a communication means or communications interface, which can incorporate any means for communicating at least data over one or more networks or to one or more peripheral devices connected to the system. Appropriate communication means may include, is not limited to, circuitry and control systems for providing wireless connections, wired connections, cellular connections, data port connections, Bluetooth connections, or any combination thereof. Numerous communications means may be utilized with exemplary embodiments of the inventive disclosure.

Peripheral devices used herein may have functionalities or constituent parts. A peripheral device may include, for example, a module for a caregiver such as a home health aide or personal care assistant. The caregiver module may be operatively disposed on a mobile device and provide a computing-device enabled connection to one or more servers. The caregiver module may include a user engagement panel, which enables the caregiver to carry out described functions of the caregiver device via a user interface. Information about a caregiver may also be provided on the caregiver device, including appointment times and locations, in addition to certain functions such as clocking in for work and inputting details of such work (e.g., a description of what care was given to the relevant patient, the time of that care, etc.). The caregiver module may include functionalities to record time information and geolocation information. Additionally, the caregiver module may require a signature or another form of verification confirmation (e.g., account number, PIN, passcode, biometric, etc.) that syncs to the patient. It will be understood by one of ordinary skill in the art that these functions may be built in to a mobile device such as a modern smartphone, smartwatch, tablet, or another external remote device. However, a geolocation identifier or clock mechanism, which may be external to the module and communicatively linked, either wirelessly or through a wired medium, may be used. The geolocation information may be used by the system or another relevant party to verify that a caregiver was at the location the caregiver was scheduled to be or purported to be, and whether it was at the correct or an acceptable time.

Certain functionalities may also be achieved by providing a patient module. The patient may use the patient module, which may be in communication with the caregiver module, another patient module, or one or more servers, while allowing the patient to contact a relevant vendor or other party. The patient module may include an application that may be cross-platformed to allow the patient to use the functionalities of a mobile device such as a smartphone, tablet, or other computing devices such as laptops or PCs. The application may also provide the patient means to access the system where the patient may access past service information, profile information, medical information, etc. The patient module and its functionalities may be relevant at one or more points in a visit. In addition to allowing the patient to contact a caregiving agency, the patient module may be used to confirm an address or a time for a visit. In addition, the patient may sign for services provided through a signature collection or other type of authentication. In certain exemplary embodiments, this information may be used as a cross-reference to data collected by the caregiver module to supplement any information that is submitted or cannot be submitted due to system error, signal error, network error, etc.

The patient's mobile device may be communicatively linked with one or more servers in order to transmit and receive information that may be necessary to communicate with other parties or to submit patient information to the system. The patient device may include, whether built into a smartphone or mobile device or connected wirelessly or through a wired medium, additional components including at least a camera, a GPS, a facial recognition system, a voice recognition system, and a clock mechanism. The camera is preferably one having location awareness, a device feature delivering information about the device's physical location to another user or application, which is commonly used in reference to mobile communication devices and cameras. Additionally, the patient module may be also communicatively linked to the caregiver module to establish a field of acceptability and verify caregiver compliance with the agency.

In addition to signature collection on the caregiver module, a patient module may allow email identification, SMS identification, or other available verification methods. These may be used individually or together in multi-factor authentication. Patient identification may also be done through physical feature recognition, such a fingerprint scan, voice recognition, facial recognition, or potentially retina scans. In one embodiment, the system may require a patient to submit a fingerprint and input a generated authentication code which may be refreshed every one to two minutes in order to submit a signature for a visit verification. Verification on both the patient module and the caregiver module for enhanced accuracy.

One or more servers may additionally access information from the patient module, use information provided by it, and potentially link it to data collected from the caregiver module as and a billing device. For example, the patient module may be used for tracking functions through inclusion of a geolocation identifier. One or more servers can request the location of the patient programmatically at predetermined intervals or upon request. Additionally, the same geolocational identifier can be used to track the caregiver to ensure caregiver compliance with the service request. A geolocational device will only function and report data if properly synced with a patient's communication device.

A server or onboard memory may be provided with the caregiver and patient modules to handle all verification requests when there is no internet or communication network available. As in such situations there will be predetermined intervals of time for sign-in for both patient and client to interact, the memory function can be configured to store these requests until a communications network is available to ensure proper billing requests for the caregiver, and to ensure that services are provided for the patient, even when offline. All of the sign-ins may be stored as data verifiable by the administrator and/or system, and then automatically updated to the central server upon the next visit where a communications network is available. This data will only correspond to each synced caregiver/patient.

Any page, photograph, report, or other submission generated by the patient module through the user engagement panel regarding a signature or any caregiver data collection submitted from the patient module may be geotagged, timestamped, and/or marked as to which patient module it may have originated from to enhance its accuracy and legitimacy. The submission may be relayed to one or more servers as raw data, and processed by the servers (e.g., properly formatted) based on billing needs. In other situations, the formatting may be to nativize the information to a format needed by a caregiving agency.

One of ordinary skill in the art will appreciate, however, that a mobile patient device may not be implemented for all types of patients. Some patients might not be willing to use and/or be unable to use a mobile device, and instead opt for a desktop computer, a fob device, a landline phone, a non-internet enabled mobile phone, or another computing device which is able to communicatively link with one or more of the agency's servers. In such cases, these preferences or inabilities may be accommodated for by the caregiver by requesting that the patient sign-in using his/her caregiver device.

Current issues facing caregiver compliance include two main issues: time tracking and location tracking. Timing issues occur when caregivers clock in during times when they are not scheduled to provide a service visit, or when administrators verify time for services for times or days when the caregiver was not present at the patient's home (e.g., due to intentional or unintentional false reporting by caregivers or unintentional error by administrators). Such false entries create issues because insurance companies are being billed for services that never took place, or for services that took place during different times. To combat this, in certain embodiments, a device syncs a caregiver and patient(s) together within a specific calendar period for which the caregiver is scheduled. At all times, the caregiver must be within a range of the patient providing services and safety supervision. A field of acceptability is established that a caregiver must stay within in order to be compliant with the service request. For different reasons, a caregiver may have to travel outside of the field of acceptability. When this occurs, the home health agency and/or the insurance company will receive an alert, and the caregiver will be given a chance to provide a reason for the non-compliance through the user engagement panel. The reason(s) may include evidence (e.g., taking a metadata enriched photograph with embedded location/time data) and/or manually inputting a reason from either a menu of predetermined reasons or a custom reason provided by the caregiver. Should the caregiver not be able to verify visitation during different intervals in the day, the onboard memory will store this data to be reviewed by the agency and transferred during the next available visit.

A field of acceptability is established by the system to enable caregivers to verify their visits upon arrival at the patient's home and/or other locations (visit verification). Predetermined rules govern the parameters of the field of acceptability. As further discussed below, in certain embodiments, the system may determine that a caregiver and a patient are within or not within the field of acceptability at different time intervals. This determination may be used to start the process of determining whether a caregiver is eligible for payment for services provided in a billing request. If the caregiver and patient are within the field of acceptability, the relevant input may be automatically adjusted to match the designated information received so that it may be accepted by the system. If the caregiver or patient is not within the field of acceptability, either may be prompted to submit one or more reasons or a photogram enriched with geolocation and time metadata. The field of acceptability may be established based on the patient's place of residence and/or other relevant location. Essentially, the caregiver's location is dynamically compared to the patient's location to verify their presence. This information may be used for reconciliation reasons for caregiver absence from the field of acceptability and may be submitted to a central server. Additionally, a caregiver may clock in or be requested to clock in at predetermined time intervals to provide verification that the caregiver is still present at the field of acceptability. The predetermined rules that govern the field of acceptability are subject to dynamic updates through analyzing the data collected.

In certain embodiments, the caregiver and the patient leave a scheduled location together, and a temporary field of acceptability may be established along any new locations or routes provided certain criteria are met, such as, for example, authentication by the caregiver that he/she requested/approved service at or associated with the new location and/or travel thereto. For example, the caregiver may take a walk with the patient for exercise purposes, accompany the patient to run errands, or other reasons where a caregiver and patient are not within the initially scheduled field of acceptability. In these situations, the caregiver is still providing services to the proper patient but at a different location. The user engagement panel allows a caregiver to communicate with the agency to explain the reason for being outside of the field of acceptability by submitting one or more reasons.

The fields of acceptability may be used to determine whether a caregiver is compliant (e.g., with respect to whether care was given according to time and/or location requirements). One or more fields of acceptability may define an acceptable input for a billing request verification. The “field” in the field of acceptability may relate to information about the visit time, such as start time, end time, or duration of service. The field may also relate to location or geolocation, such as a defined area considered to be an acceptable geolocation input. At relevant points during a service visit, it may be determined that a caregiver is within or not within the field of acceptability if the caregiver and patient cannot be verified together as a pair at the allowed location. The parameters which govern the field of acceptability may be predetermined to reflect any reasonable distance, time, or other parameters. Herein, “designated” may refer to information input and received during a scheduled service visit (e.g., a time period entered as the intended visit duration). In contrast, “actual” may denote information tracked through a module or other enabled device (e.g., a location where a service visit may be indicated as having taken place). Designated and actual times and time periods may include one or more points of comparison to determine whether the caregiver was in the field of acceptability.

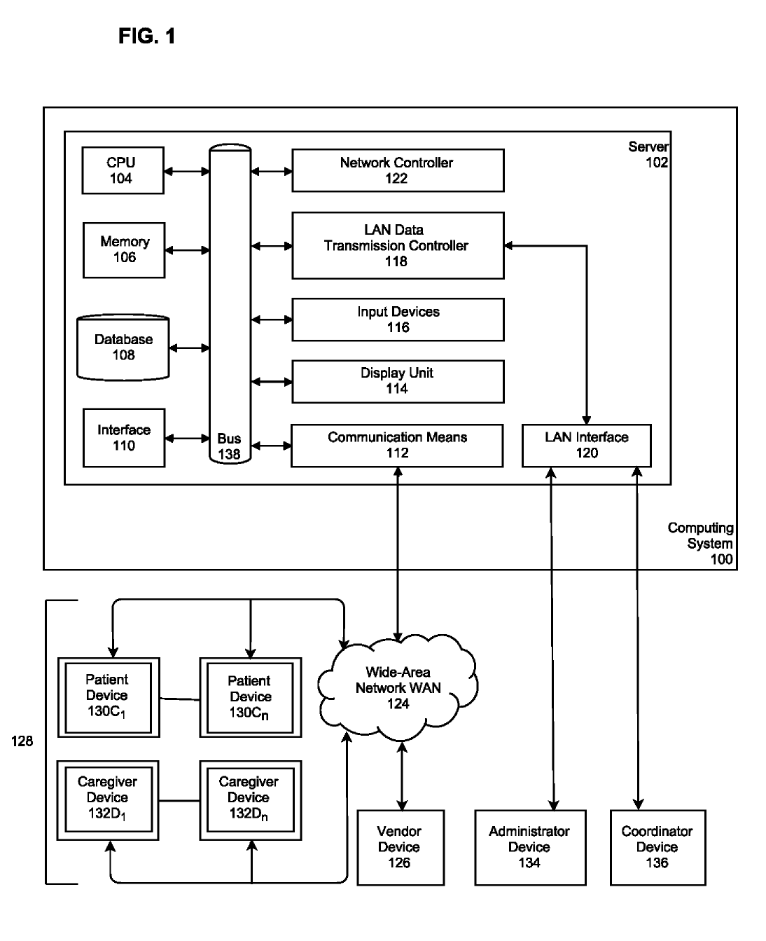

The systems and methods described herein are best understood with reference to the following drawings, which are described in detail below. Referring first to FIG. 1, illustrated is a diagram of an exemplary computing system 100 and a plurality of peripheral computing devices 128. A combination of hardware and software operate on the plurality of computing devices 128 and computing system 100, generally with one or more connections to wired or wireless wide area network (WAN) 124 (e.g., the Internet), and are incorporated with local devices through local area network (LAN) interface 120. Computing devices 128 can be a wireless mobile hardware device having software capable of communicating information to other mobile devices or computer systems, determining the location of that device with geographical position location capability (e.g., through triangulation of a cell system, GPS, specifically input by a user, etc.), and connecting to a private computer network or public network such as the Internet through a network.

Computing system 100 can include, for example, server 102 including central processing unit (CPU) 104, memory unit 106, database 108, interface 110, communication means 112, display unit 114, one or more input devices 116 (e.g., keyboard, mouse, etc.), LAN data transmission controller 118, LAN interface 120, network controller 122, and internal bus 138. As shown, the system may be connected to a data storage device, such as, for example, a hard disk disposing one or more databases 108 via a wired or wireless link. Computing system 100 can include one or more servers configured the same or similar to server 102 shown in FIG. 1, or one or more servers configured in a different manner, which may dispose different hardware or software. For example, computing system 100 may comprise multiple servers hosted in multiple spaces such as data centers or server farms.

Computing system 100 may be configured to communicate with network service(s) coordinated through communication means 112, which may include any approach for communicating data over one or more networks or to one or more peripheral devices.

Communication means 112 may include, but is not limited to, circuitry and control systems for providing wireless connections, wired connections, cellular connections, data port connections, Bluetooth connections, or any combination thereof, and the means may include devices enabled to communicate using such communication approaches. One of ordinary skill in the art will appreciate that there are numerous approaches to communications that may be utilized.

Server 102 and computing system 100 may be communicatively linked, through communication means 112 and WAN 124, to peripheral devices such as computing devices 128, vendor device 126, administrator device 134, and coordinator device 136. Computing devices 128 may be configured as one or more patient computing devices 130C1-130Cn or caregiver computing devices 132D1-132Dn. Computing devices 128 may be devices (e.g., smartphone, smartwatch, etc.) which allow a user (e.g., patient, caregiver, etc.) to interact with the computing system 100. Any number (e.g., 1, 2, 3, . . . n) of caregiver devices 132D1 . . . 132Dn, or patient devices 130C1 . . . 130Cn may be used in conjunction with computing system 100.

It will also be appreciated that remote computing devices 128 may additionally or alternatively include non-smartphone(s) (i.e., traditional cellphones, landline telephones, etc.) that may connect with computing system 100 through a network, which may be telephone communication network without the Internet. In such embodiments, a patient's voice request for home healthcare service is transmitted to the central server where voice recognition is performed and the healthcare request is processed. The system then responds automatically with the caregiver details and estimated time of arrival information. The central server may preferably work in conjunction with a database to store previous service information, such that it may later utilize the data to match the patient's voice to a log of previous service requests by the patient. An automated response by an interactive voice response (IVR) system may inquire as to whether it is the same service request type that was most previously completed. The patient may be given a chance to choose “yes” or “no” or to select from a number of options, so that the request can be processed automatically. Alternatively, the telephone system may prompt the patient to verbally provide new service request details. Once this is processed, the system will automatically respond with the caregiver details, estimated time of arrival (ETA) information, etc.

The system and method may further provide a multi-level IVR system where callers are asked to choose from a series of audio prompts (e.g., “Press 1 for . . . ”), and then based on the caller's selection, are given a series of audio prompts to choose from, and so on. Callers may continue to be routed through the system until the necessary information is received and/or provided. Multi-level IVRs are customizable and can handle many prompts and levels of the IVR the callers must go through until their inquiry or request is properly handled by the system or they are properly routed.

Computing system 100 may have more than one server 102 in a distributed structure with support from data centers that may be located anywhere around the world. These implementations may be communicatively linked and cross-platformed so that a user on a mobile device (e.g., smartphone, tablet, etc.) or stationary device (e.g., desktop computer, landline telephone, etc.) may be provided with information relevant to their service request (e.g., electronic map displays, indicators which display travel times, routes, pricing information, profile information, settings information, level of familiarity, etc.). The term indicator as used herein is a means to transmit or display information related to services to a patient or to a service provider or both in a simple, fast, and convenient way. Features of the systems described herein can be implemented through computing devices that allow for method steps to be processed and output by a processor. Server 102 preferably coordinates user interfaces and interacts with database 108. Each user, depending on that user's role and needs, may be provided with a different functionality.

Server 102, through a server interface, may receive patient input information, location information, and service request information to configure content, as well as the information from the caregiver (e.g., location information, limitation information, historical information, etc.). As discussed above, server 102 can send information to one or more computing devices through server interfaces, and information can be output to a display of the computing devices. Such content can include features which are region-specific if particularly relevant regional information exists, especially with respect to service request mapping or routing.

The service request information received through the server interface may be stored by the computing system 100 in database 108, and may include, for example, the status of service requests, the status of service request acceptances by caregivers, the reasons from caregivers for cancelling service requests, the histories associated with assigned service requests, operation logs of coordinators, etc. The content/timestamps of notifications and confirmation statuses may also be recorded in system logs, and this information can be checked by the administrator of computing system 100. It will be appreciated that this is not an exhaustive list of the operational service request information that the system may record.

The data stored in one or more databases 108 of computing system 100 can be continually updated with all user information discussed herein and analyzed in accordance with the various methodologies discussed herein to enable efficient booking of home healthcare services. Every time computing system 100 gets an input/request from a patient, a caregiver, a coordinator, or other user, computing system 100 can first open a safe access channel with database(s)/database center and then send out query sentences through the access channel to a database management module. If a relational database is utilized, then the data tables may have one kind of relationships, such as one to many relationships, many to many relationships, and one to one relationships with other data table(s). Based on the relationships between data tables, the database(s) management module can exactly follow the query sentences and find the specific data table(s) by using ID(s), table names and column names of the tables with/without joining two or more data tables together. If a non-relational database is utilized instead of data tables, with the data stored in key-value pairs, then the database management module can exactly follow the query sentences and find the specific data by using keys that query sentences provide.

Computing system 100 can access all information stored in one or more databases 108, which may include rules and procedures data, caregiver data, administrative data, group data, patient data, map component data, and any other data relevant to implementation of computing system 100. Rules and procedures data can include system price, promotion setting rules and procedures, as well as rules and procedures for indicators, referrals, payments, service requests, system management, system log, system analysis and optimization, etc. The map component data can store map data for service requests that are identified by the GPS and location-based services (LBS). The GPS and LBS data can determine the location of the computing devices in different ways, such as, for example, through receiving location-based resources. Caregiver data can include caregivers' profiles, such as personal data including a photo of the caregiver and years of his/her experience, certification levels, gender, country of origin, and language abilities.

Comprehensive database 108 may also store the details of service requests for each particular caregiver for future reference. Database 108 can also include data in the caregiver's profile that may include such information as a caregiver's limitations related to zip codes, time, location, and price, as well as service data and records. There are numerous methods of providing databases and data storage media for the organization and retrieval of specific data, and exemplary embodiments of the inventive disclosure are contemplated for use with any appropriate database(s) or storage means. In some implementations, database 108 can be located and accessed remotely. Further, while referenced as a database, it will be appreciated that database 108 may include a data storage medium, whether structured or unstructured, relational, or otherwise. Database 108 may be dynamically updated as services are booked and completed. Database 108 may store an index of each service request that has been requested and completed, which can be retrieved for reference if needed at any time, as well as store the details of service requests or billing requests organized for each particular caregiver, patient, vendor, or service provider for future reference.

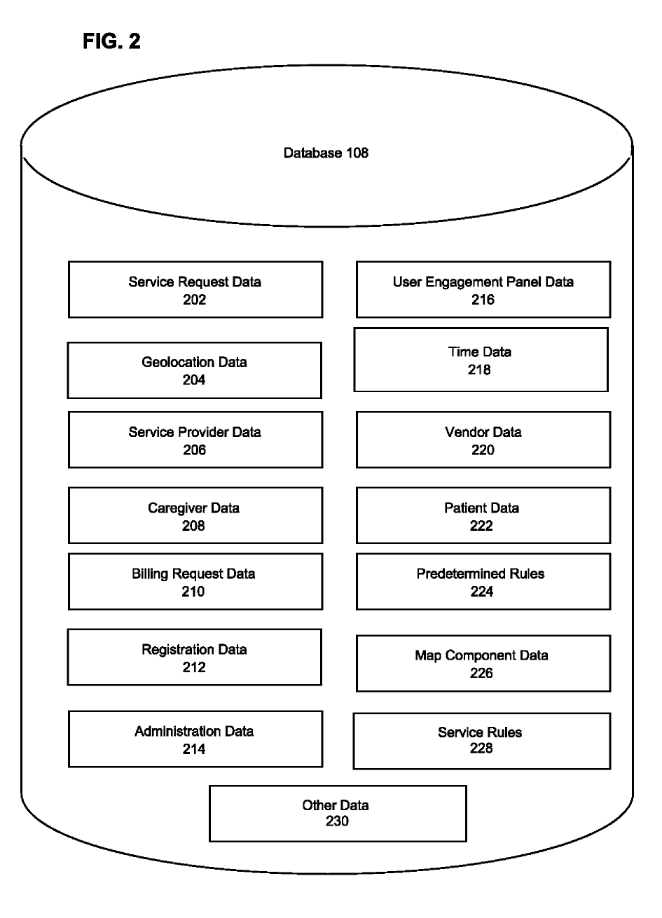

As depicted in FIG. 2, database 108 may contain service request data 202, such as when and from where a service request was made, who made the service request, who provided the service request, the name of the caregiver (e.g., doctor, aide, therapist, etc.) the patient saw, etc. In addition, any data provided through user engagement panel 314 (user engagement panel data 216), may be stored in its own category with subcategories thereof. Database 108 may also store geolocation data 204, such as the designated and actual geolocations as well as the buffer parameters and/or the field of acceptability for each geolocation. In addition, database 108 may store time data 218, also regarding the buffer parameters and/or the field of acceptability relevant for each time adjustment. Database 108 may also store service provider data 206, vendor data 220, caregiver data 208, and patient data 222. This data may reflect any and all records each user may generate, which may include profiles, service request history, billing request history, etc. Database 108 may also store billing request data 210. Database 108 may be configured to categorize and store the predetermined rules 224 that define the field of acceptability. Those rules may be associated with certain service requests, one or more locations or times, one or more vendors, one or more patients, etc. In this manner, one or more servers 102 can retrieve the applicable predetermined rules from database 108 when needed. In addition, database 108 may store registration data 212 of all users. Additionally, database 108 may store map component data 226 which may be queried for use as a GIS 102 map layer or for other relevant purposes. Map component data 226 may contain data retrieved from a third-party geocoding application program interface (API) such as GOOGLE MAPS. Further, database 108 may be configured to store data related to system administration in administration data 214, service rules 228, as well as any other data 230 relevant to the electronic billing verification.

One of ordinary skill in the art will appreciate that database 108 can sync dynamically so that whenever changes or updates in data blocks are made, server 102 and database 108 dynamically update the data accordingly to reflect the latest changes. Additionally, at least one backup database may be utilized to back up a primary one of databases 108 in case of data loss in the primary database 108. One of ordinary skill in the art will appreciate that database 108 components may vary from the ones depicted herein.

Alternatively, computing system 100 may use a set of databases or data storage mediums to provide and maintain a prescheduled service application in order to dispatch a compatible caregiver based on a patient's preferences and needs. Databases 108 may contain several data categories or groupings. Sections of database 108 may be independent or synchronized in order to retrieve information from both sections at the same time. Such data can include predetermined rules data 224, procedures data, administrative data 214, patient data 222, caregiver data 208, group data comprising base data, company's data, or group of individuals' data, and other types of data such as data relating to user types. All historical information may be categorized and stored in and retrieved from database 108. Historical data can be tracked in part by assigning a tracking number, service ID number or service ID corresponding to each service request to help computing system 100 refer back to the service request. Information categorized with this identification may include the type of service request, who requested and carried it out, where it took place (e.g., zip code, borough, county, city, state, etc.), the cost of the service request, when and how payment for the service took place, etc. All information regarding a patient's or caregiver's preferences or limitations, pricing, and other customizable information can be stored within database 108.

The service request information stored in the database 108 may include, for example, a service request ID, relevant caregiver information, relevant patient information, requested visit location, actual visit location, visit start time, visit end time, distance, duration time, status, prices, insurance company, etc. Even if a patient does not have a smartphone or use the application which is in communication with the system, this will not adversely affect the functioning of the system as methods which circumvent a patient not having access to the system may be utilized. For example, a coordinator may update such patient on the status of his/her service request or on the location of the caregiver. The coordinator can provide the patient with the most current information, as a start button functionality discussed below allows a caregiver to instantly connect with the coordinator.

The relevant service information may include information such as first name, last name, username, email, password, phone number, date of birth, gender, country of origin, caregiver type, affiliated company name, provide wheelchair, language, signature, and general profile. The relevant service information may also include insurance information such as liability status, insurance status, insurance provider, insurance start date, and insurance end date.

Any backup databases related to database 108 may also be updated accordingly to reflect the latest changes. Such information can be organized or structured in a manner allowing for effective sorting and retrieval. The information can be stored in a non-relational or unstructured manner. One of ordinary skill in the art will appreciate that numerous methods for providing, storing, and organizing data in database 108 or other data storage medium may be utilized in accordance with the exemplary embodiments of the inventive disclosure discussed herein. Additionally, it will be appreciated that at least one backup database may be utilized which backs up the primary database frequently in case any data is lost from the primary database.

Additional data may be input into database 108, including, but not limited to, locations where patients traveled to, other transaction data and details, historical data, insurance policy expiration dates, caregivers' license expiration dates, or any combination thereof. This data may also include information relating to indicators and the display thereof. By way of example, the data may include the service requests that all patients or caregivers have completed in a certain area, such as one or more streets, zip codes, town, city, borough, county, state, or any other region defining feature, or how many times a patient and a caregiver have been paired.

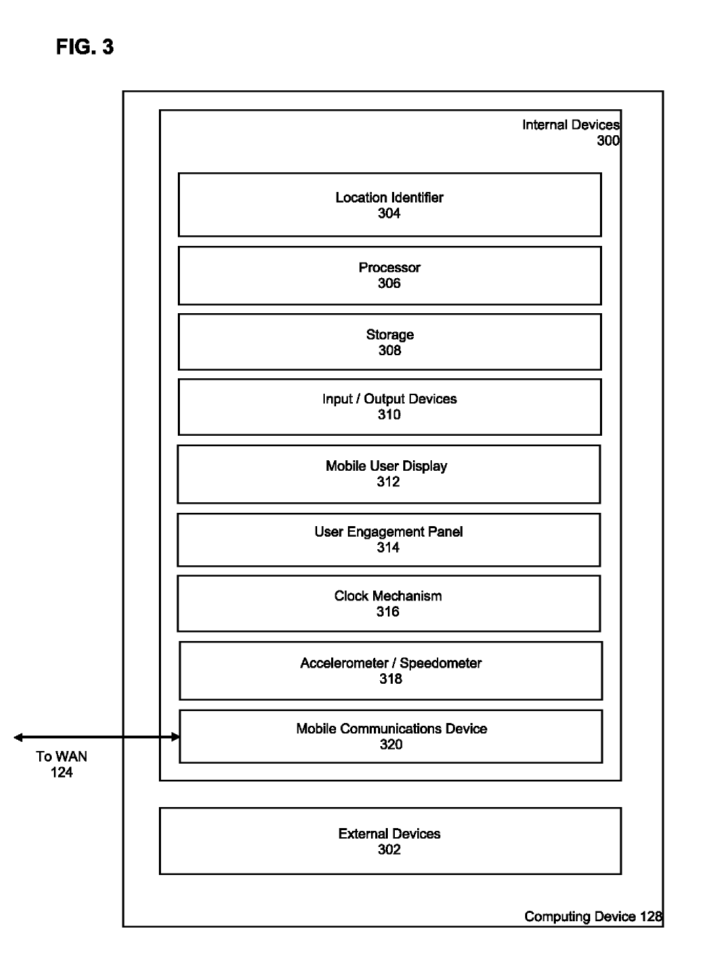

Turning now to FIG. 3, shown is a diagram illustrating various components of an exemplary embodiment of computing devices 128. As discussed above, computing devices 128 can be used by patients (e.g., via devices 130C1 . . . 130Cn) or caregivers (e.g., via devices 132D1 . . . 132Dn) or both, or even service providers, coordinators, administrators or vendors, and may be in communication with various components, tangible or intangible, of computing system 100. Computing devices 128 may incorporate various internal devices 300 and external devices 302, and may utilize mobile communications device 320 for receiving voice, text, and/or data for connecting to computing system 100 such as over WAN 124, and location identifier 304 such as a GPS receiver or GPS unit for identification of a past, present, or future location. An application, a map component, map data, and location identifier 304, such as, for example, a GPS module or other circuitry for providing LBS data may be integrated into one or more of computing devices 128 for certain location identification functions. Location identifier 304 may identify a location of computing devices 128 in different ways, such as, for example, through receiving location-based resources. One of ordinary skill in the art will appreciate that there are numerous approaches for providing location identification and location-based services. A GPS-enabled system or device allows tracking components to identify the location of computing devices 128. For example, location identifier 304 can be instantiated through processing received GPS data from location-based or geo-aware resources of computing devices 128. Additionally, location identifier 304 can receive GPS data from other applications or programs that operate on the computing device using one or more APIs. The application can use location information to cause a user interface component to configure a user interface framework.

In preferred embodiments, a patient can access a patient module such as computing device 128 operatively associated with computing system 100 to input a service request. If, however, any entity requests a service which is deemed incompatible with the system (e.g., based on data received from location identifier 304 regarding the location of one or more caregivers and/or caregiver limitations in database 108), then a coordinator may be notified.

Computing system 100 may be configured to generate a notification to patient device 130 when a caregiver comes within a region that is a certain distance (e.g., one or two miles) of a patient visit location designated in a service request. Such location-based services, facilitated by location identifiers 304 in caregiver devices 132D1 . . . 132Dn, allow for efficient routing of caregivers based on point-to-point directions. Caregiver devices 132D1 . . . 132Dn may each include an interface component which provides location information gathered by location identifier 304 and passes such location information to an interface component on patient device 130C1 . . . 130Cn via WAN 124 and server 102. Caregiver devices 132D1 . . . 132Dn may also include radio-frequency identification (RFID) technology, or other similar identification device or means.

Location identifier 304 can include a GPS-enabled system or device whose tracking components identify the location of patients who are making service requests and caregivers who are looking to provide service. While the inventive disclosure is primarily discussed as incorporating or utilizing GPS technology, other tracking services such as China's BeiDou network, Russia's GLONASS service, India's IRNSS having the operational name NAVIC, Japan's QZSS, and Europe's Galileo network, etc. may also be employed as or part of the location identifier in accordance with exemplary embodiments of the inventive disclosure. Computing system 100 may include an application manager which, based on a patient's current location or service location, may cause a region-specific patient interface feature to be output by a patient interface component on mobile user display 312. A region specific to a patient can include the patient's current location or a service location in which the patient wishes to preschedule service. As used herein, the service location is the location initially designated as the location at which the services are to be provided, which may include a certain area or region around a geolocation point as determined by certain rules. The region may be identified by zip code, city name, metropolitan area name, etc., in which computing devices 128 are currently located, and may be an area having a certain distance or radius from the patient's current location (e.g., one mile, five miles, etc.), or may be an area specifically partitioned from other areas. Region-specific information about the prescheduled service may be provided in part by a system which supplies caregiver location data using location identifier 304. It will be appreciated that use of location related preferences or limitations may depend in part on GPS-enabled devices. By cross-referencing data, the prescheduled service systems described herein can identify specific locations (e.g., stores, restaurants, apartment complexes, venues, street addresses, etc.) proximate to and/or located at an identified location and provide this specific location information as location data (e.g., as part of caregiver and patient pair map component data 226).

Processor 306 is preferably provided for executing software or a set of instructions on computing devices 128. Computing devices 128 may also contain storage 308 such as random-access memory (RAM) or flash storage. Input/output (110) devices 310 can be used to connect computing devices 128 to other system implements, depending on the available functionalities of computing devices 128. For example, a caregiver may use an in-vehicle navigation system, which might not have a camera, while a smartphone may have a built-in camera. In this situation, a camera may be included as an input for the in-vehicle navigation system. Other I/O devices 310 may include a scanner, a microphone, and/or a speaker. Computing device 128 may also include mobile user display 312 to receive and display to a user notifications and/or other data received from computing system 100. Mobile user display 312 may, for example, be an electronic touchscreen display configured to provide user engagement panel or interface 314 in accordance with various methodologies of the inventive disclosure. Computing devices 128 may also utilize internal clock mechanism 316 to determine the time the devices were at a given location. Accelerometer/speedometer 318 can also be provided as part of or in communication with computing devices 128 to measure speed, acceleration, directional changes, etc.

User engagement panel or interface 314 displays various content based on user selections and preferences. It will be appreciated that one or more components of computing devices 128 may be combined to provide user features specific to user selections and user locations. These selections can be displayed to the user, and the user can utilize user engagement panel or interface 314 to interact with displays of certain information. For instance, user engagement panel or interface 314 can correspond to a program downloaded onto a smartphone or other portable computer device such as a tablet computer or personal digital assistant (PDA). A user can download and install the application on one or more computing devices 128 and register with the system. Pre-programmed features of computing devices 128 may be utilized based on certain protocols or methods of integration of basic components, such as servers, databases 108, mobile end applications, web portals, network settings, etc.

All types of users can be registered and entered within the system for the purpose of activity tracking. Registration may be performed through means such as assigning a user ID to each user such that system functionalities can only be accessed when a user ID is entered. Additionally, the device the user employs to access the system may be tracked by an Internet Protocol (IP) address, and system activity may be tracked with timestamps or similar means and stored in database 108. In this manner, a system administrator can track not only who is accessing the system, but also from which device, the location of the device, and the time of such access. Such capabilities allow coordinators to track activity, and if an error occurs, such as entry of a wrong address on a service request, then the cause of the error can be easily diagnosed and addressed. It is currently a drawback in the industry that such errors cannot be detected and located, especially when a coordinator does not want to admit the error. It will be appreciated that such functionality also provides a means for added security. Any service request entered from an unauthorized computer can be ignored. Unless computing device 128 is given access permission, it cannot access certain functionalities restricted to registered users. Allowing a caregiver system to be run in part by coordinators allows for increased flexibility and executive function if necessary, as exceptional situations can arise which require human judgement.

User engagement panel or interface 314 can be, for example, a homepage, access to a dispatch portal (for caregivers), a service requesting module (for patients), an access interface to database 108, or a way for users to access one or a combination of any of the features described herein. The system can retrieve a user's information and other data that is stored in database 108, which may be provided locally and/or remotely. One of ordinary skill in the art will appreciate that numerous additional user interfaces are contemplated for use with or in place of user engagement panel or interface 314.

Patient devices 130C1 . . . 130Cn may each utilize a patient interface to display various indicators on maps showing geographic information. Each indicator can signify, for example, differing patient information or patient inputs received by computing system 100 from the patient, a vender, or any application which takes a service request from the patient. Patient devices 130C1 . . . 130Cn can also each contain application features adapted to dynamically synchronize content based on patient selections provided via a patient input. User engagement panel or interfaces 314 on patient computing devices 130C1 . . . 130Cn can include, but are not limited to, a home page patient interface, a service request panel used for patients to identify the details of service requests, preference details, etc., a summary patient interface, a location search patient interface, a confirmation page interface, or a combination of any of these features. By way of example, if a patient's current location is different from an originally requested visit location, then the patient can manually preschedule a new visit location that is different from the current location stored in computing device 128 or computing system 100.

A start button functionality can be provided as part of computing device 128 on, for example, one or more of caregiver devices 132D1 . . . 132Dn. Display 312 of one or more mobile caregiver device 132D1 . . . 132Dn may display relevant information to a caregiver about the service queue, starting with the next service in the queue, where the details of that service are shown, such as the visit location and visit start time along with the destination and the scheduled visit end time. The caregiver can then click ‘Start’ (e.g., a physical push button or via a touch screen interface on caregiver device 132D) in order to let a dispatch or administrator know that he/she has begun the service and is on the way. Mobile user display 312 may also show a list of the remaining services in the queue with limited details which may be expanded or viewed later. This Start button functionality helps address current drawbacks in communication between various parties as coordinators can easily identify which service requests are underway. Additionally, the Start button provides a means by which a patient who has scheduled a second service can let a caregiver know the status of the related appointment. In a traditional system run by telephone, the current location of caregivers and patients may be unknown. As a result, coordinators have no choice but to operate by guesswork if they cannot easily reach a caregiver or patient while coordinating a service request. When a caregiver presses a start button at the beginning of each service of a service request, the system can record the status in database 108. Such functionality will also make tracking easier if the duties of a service request are handled by more than one caregiver.

It will be appreciated that the systems and methods disclosed herein provide functionalities allowing for a much smoother and more efficient process overall than those of conventional service methods. Pressing the Start button may trigger a series of events which may be carried out with the help of various software and hardware implements. Pressing the Start button may, for example, cause the caregiver's location to be relayed to a third-party mapping and navigation service, which may configure various routing options and the ETA for the caregiver on each route based on the caregiver's current speed and the distance associated with each route. This information may be relayed to a dispatch, a patient, and back to the caregiver, and may be provided in real-time.

The user interface may also include a Start button which triggers a series of events in database 108 regarding data storage, where the geolocation of the caregiver is tracked by location identifier 304 as part of the records associated with a service request, the patient, the caregiver, etc. Without this Start button functionality, GPS devices may still be able to detect a caregiver's current location and heading. However, when providing services, the caregivers will have a long list of scheduled services, and without a caregiver actually confirming that he/she is underway to provide service to a particular patient, there is no way to be sure that the location which the caregiver seems to be heading towards is the patient's visit location. As a result, the Start button is a powerful feature that can provide coordinators and other parties with an instant update on the caregiver's real-time status.

Other functionalities may be of use in Non-Emergency Medical Transportation (NEMT), including functionalities provided for users on the clinic end. A clinic operator such as an employee of the clinic may be able to manage (e.g., search/add/delete/modify) appointments affiliated with the clinic, and by using the functionality of a start button, notify other employees of the clinic and patients of any changes. For example, the clinic operator or a patient may be able to notify other users that an appointment has begun by pressing the Start button on their end. If, for instance, the appointment began later than scheduled, the system may be able to make any necessary changes, such as shifting an appointment to a different time or notifying patients of how the changes will impact their appointments. Additionally, a caregiver who has been assigned to pick-up the patient at the end of the appointment may get a real-time notification about the status of the patient, such as “Checked In,” “Seeing Doctor,” “Almost Finished,” “Finished,” etc., which allows a caregiver to be ready for any changes in the scheduled visit start time.

Patient devices 130C1 . . . 130Cn may be configured to allow a patient to manually supply location inputs by entering an address (e.g., street number, street name, city, state, etc.) or by manipulating and moving a service location graphic/icon on an electronic map display on a portion of a patient interface. In response to such patient selection, the service application running on one or more of patient devices 130C1 . . . 130Cn can provide the location data to computing system 100. Once the Start button is engaged, computing system 100 can calculate an ETA, and a caregiver may be provided with potential jobs through this interface, where queries can be displayed temporarily for the dispatch of a service. A caregiver module can facilitate enabling the interface on a mobile device. In the event that a patient cannot sign for a service, computing system 100 may depend on the caregiver to collect a signature on his/her phone from a patient.

In preferred embodiments, system 100 dynamically updates and stores any changes to a service request before or during the start thereof, or any updates on the status of the service request and displays these changes in real-time, both in a web portal for the coordinator, and in a caregiver interface on caregiver device 132 associated with the caregiver assigned to the service request. For example, if a patient cancels a service request or needs to change the visit start time or location, the patient can enter this information into system 100 via patient device 130. The new information is stored in database 108. The web portal of the coordinator updates, and a notification of the change is immediately sent to the caregiver associated with the service request via caregiver device 132. The caregiver can then preferably access the same information about the service request displayed in the web portal of the coordinator. Additionally, in preferred embodiments, any new information about the patient (e.g., phone number, email address, a change to preferences, etc.) entered prior to the service request can be communicated to the web portal of the coordinator and the user interface of caregiver device 132 of the caregiver assigned to the service request. Preferably, only relevant caregiver devices are updated with new patient information (e.g., caregiver devices associated with caregivers involved with the patient's service requests).

When a caregiver presses the Start button at the beginning of each service or portion of a service request, the service status can be updated in the web portal of the coordinator and the patient device 130 of the patient. It will be appreciated that a patient can thus view an estimate of the arrival time of his/her caregiver in advance thereof. Such functionality will diminish or eliminate the workload of a coordinator as he/she will not need to field phone calls regarding changes to service and will not have to call caregivers. Those skilled in the art will appreciate that these functions are merely examples, and that other functionalities of the caregiver's interface may be utilized.

External devices 302 (i.e., additional mobile devices, tablets, laptops, or other computing devices) can connect to computing devices 128 through a wired or wireless connection. It will be appreciated that these external devices 302 may include any device that can provide additional or enhanced functionalities to computing devices 128, whether computing devices 128 is a mobile device such as a tablet or smartphone, an in-vehicle navigation system, or another type of computing device.

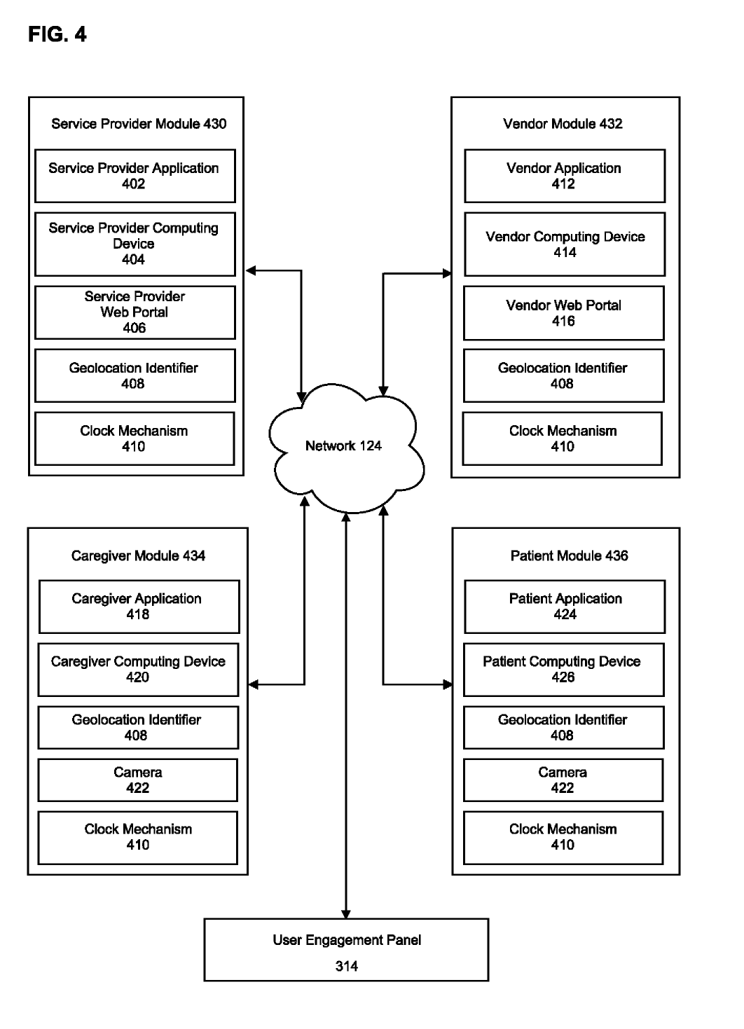

Shown in FIG. 4 is a schematic diagram further illustrating system components for home healthcare electronic billing verification and caregiver tracking in accordance with an exemplary embodiment of the inventive disclosure. Service provider module 430 may include service provider application 402, which may be software running on service provider computing device 404 such as a desktop computer or touch screen device or include a keyboard and/or a mouse or other input devices. A service provider (e.g., a human user such as a biller who is employed or affiliated with the service provider) can enter commands and information into the system through service provider module 430 running on service provider computing device 404. Other input devices can include a microphone, tablet, smartphone or other mobile device. Additional functionality may be provided through a scanner, etc. These and other input devices are connected to one or more servers through an interface. Service provider application 402 may be in the form of different apps such as desktop app, Android apps, iOS apps, or Windows OS apps, etc., and may run on a computing device, such as a mobile device. The application also may be maintained by maintenance specialists with related monitoring and/or management tools and may be upgraded by engineers with related experience and skills. The server may also allow for a backup on service provider module 430 for added security. Service provider module 430 in part allows a service provider to connect to service provider web portal 406, in which certain billing related information such as pending billing requests, service requests, etc. may be made available. Service providers may be provided features that are unique to their job functions. Service providers may be provided with much more information than caregivers to have comprehensive information regarding the services. For example, information provided for a service request may include available service information for various caregivers, the current and future planned locations of the caregivers, the directions of any vehicles the caregivers are driving, etc.

Caregiver module 434 may include caregiver application 418, which may be carried out on caregiver computing device 420. Caregiver module 434 allows tracking components such as geolocation identifier 408 (e.g., GPS receiver or GPS unit) to identify the geolocation of a caregiver, where geolocation identifier 408 may be built into a mobile device or it may be part of a specially programmed billing verification device. In other exemplary embodiments, geolocation identifier 408 may be mounted in a vehicle operated by a caregiver and may communicate through network 124 with one or more servers 102 with means for geolocation tracking and/or processing. Caregiver module 434 may also use camera 422 to take pictures, and clock mechanism 410 to obtain timestamps. These may be part of caregiver computing device 420, such as built in to a smartphone, or they may be input devices or potentially functionalities provided by an additional specially programmed billing verification device. Camera 422 may allow the caregiver to take a photo of a certain location, and that photo may be image processed. The photo may be processing using image-recognition technology to cross-reference the photo with known location images.

For example, a caregiver/patient arriving at the doctor's office for an appointment may be able to take a photo of the entrance into the doctor's office, which may be recognized by the system. The system may have stored images or video recordings of that same building entrance, and that provided photo can be further examined to provide an authentication. Those images or video recordings may be stored within database 108, which can be queried for its content. In addition, those images may be provided through an API such as the APIs of GOOGLE MAPS, a mapping and turn-by-turn direction application provided by Google, a subsidiary of Alphabet Inc., which feature street images that correspond to correct addresses, which may also be mined for authentication purposes. This functionality may be useful in the case of caregiver module 434 having communication problems with one or more servers 102, or problems otherwise maintaining a stable connection. In such a case, this may provide a way to compensate for certain technological failings in connection networks.

Caregiver application 418 may additionally allow the caregiver access to a web portal or other means of data input and/or system access. This may be for a caregiver to submit a billing request, which may go to the service provider. In an exemplary embodiment of the inventive disclosure a billing request may go indirectly or directly to a vendor. Indirectly may mean that the service provider first reviews and accepts it, then submits it to the vendor. Directly may mean that it is submitted directly to the vendor. Additionally, a caregiver may be provided with potential jobs through caregiver module 434, where queries can be displayed temporarily for the dispatch of service requests. Caregiver application 418 may also allow specific communication functionalities with other independent apparatuses within the module or externally with one or more servers 102. Through one or more servers 102 or user engagement panel 314, a caregiver may interact with a web portal or a service provider, where it is important that the caregivers are able to contact a service provider in the event of a case review, appeal, or other situation. Further, in the event a patient cannot sign for a service, the system may depend on the caregiver to collect information to confirm or verify the service request, such as a fingerprint, voice confirmation, or verification information from the patient on caregiver module 434. This may be in a case, such as a patient forgetting his/her module, leaving it at home, technical difficulties, etc. This is not an exhaustive list of the different functionalities caregiver module 434 may include, nor is it intended to be such.

The vendor may use vendor module 432, which is in communication with one or more servers 102, to access the system. Vendor module 432 may include vendor application 412. Vendor module 432 may also integrate vendor computing device 414 to implement vendor application 412. One of ordinary skill in the art may appreciate, however, vendor application 412 may be programmed to differ from service provider application 402 or caregiver application 418. In addition, a vendor that may be a business might use an internal computing network, where vendor application 412 may be designed to run on such network and on one or more vendor computing devices 414. Vendor computing device 414 may also give a vendor access to vendor module 432 through vendor web portal 416. Vendor module 432 may be configured to send a service request to be received at one or more servers 102 or one or more processing units 104. This service request may be then transmitted to service provider module 430 or caregiver module 434.

The patient may use patient module 436 to access certain system functionalities. There may be patient application 424 which is run on patient computing device 426. Patient module 436 may also include geolocation identifier 408, camera 422, and clock mechanism 410, among others. According to an exemplary embodiment of the inventive disclosure, some functionalities may be achieved by providing a patient module. The patient may use the patient module, which may be in communication with one or more servers while allowing the patient to contact a relevant vendor or other party. The patient module may include an application, and the application may be cross-platformed to allow the patient to use the functionalities of a mobile device such as a smartphone, smartwatch, tablet, or other computing devices such as laptops or PCs. The application may also provide the patient with means to access patient module 436, where the patient may access past route information, profile information, medical information, etc. The patient may also use patient module 436 to transmit confirmation information confirming completion of the service request by the caregiver, specific aspects or segments of the service request, and/or particular routes utilized to travel between two locations associated with the service request. This information may include a signature, a PIN, a passcode, a fingerprint, biometric data, a timestamp and/or location data.

It is to be understood that while service provider module 430, caregiver module 434, vendor module 432, and patient module 436 may include similar elements (such as a specific application or may contain elements such as geolocation identifier 408, camera 422, clock mechanism 410, etc.), these elements need not be identically implemented within each module. Geolocation identifier 408 and clock mechanism 410 may be used to, respectively, ascertain the location of system activity, in addition to obtaining a timestamp for that system activity on service provider module 430, vendor module 432, patient module 436, or caregiver module 434. This may be of use in potential audits or as a security measure in some cases, especially if either module is being used on a mobile computing device.

One or more servers 102 may also provide access to user engagement panel 314, which may be accessed by a user through one or more modules connecting to user engagement panel 314 through network 124. User engagement panel 314 may be a user interface (UI) element. One or more servers 102 may access database 108 or one or more databases and display relevant data within it on the user engagement panel 314. One or more servers 102 may access all information regarding a certain service request or billing request and display it. User engagement panel 314 may be provided through such means as an online forum, message board, or other type of website that allows for the online discussion of relevant topics. User engagement panel 314 may be accessed on a web browser or provided as part of a user's application or as its own application that can be run on one or more computing devices. The data on user engagement panel 314 may be accessed generally, though it potentially may be redacted for private information depending on who may be viewing it. In other instances, it may be presented in whole to certain users, such as the user the information regards.

A caregiver may access user engagement panel 314 through caregiver module 434, where the caregiver can upload a photograph, submit the reasons he/she was not within the field of acceptability, etc. On user engagement panel 314, photographs that a caregiver has uploaded may be displayed, and those photographs may include metadata with geotag information or a time stamp associated with the time the photograph was taken as well as metadata linking the photograph or other evidence to the computing device from which it was generated. For example, a digital image may include metadata that describes the means of creation of the data (e.g., the mobile phone or computing device which generated the digital image), the time and date of its creation, the creator or author of the data, the location where the data was created (e.g., the specific computing device, where on a computer network, etc.), the file size, how large the picture is, the color depth, the image resolution, the shutter speed, and other data. User engagement panel 314 can also be accessed through service provider module 430 and vendor module 432, where a user may access information for verification. From user engagement panel 314, an authorized biller that may be a service provider or a vendor may review case facts and other information and may be able to contact one or more caregivers to verify the billing request.

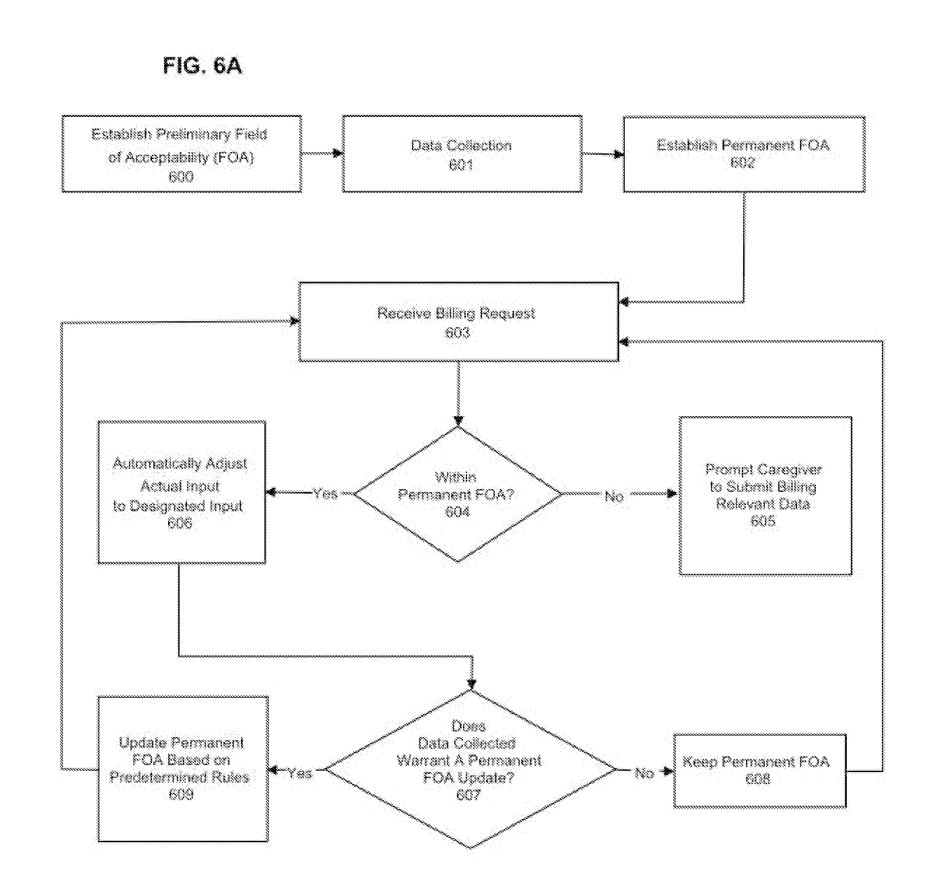

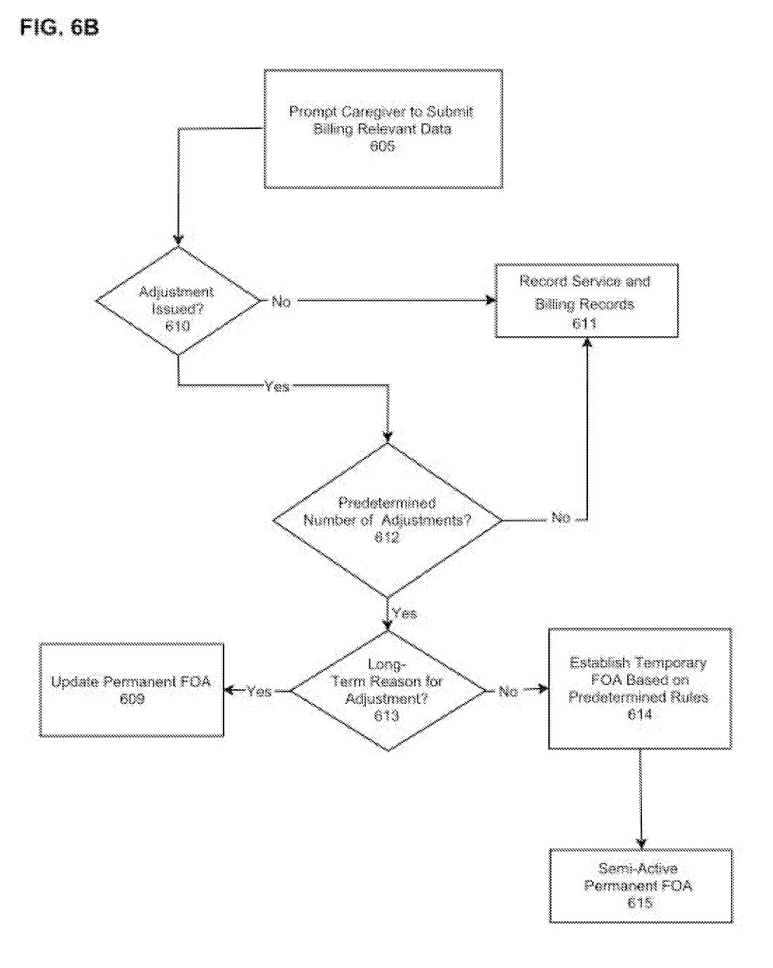

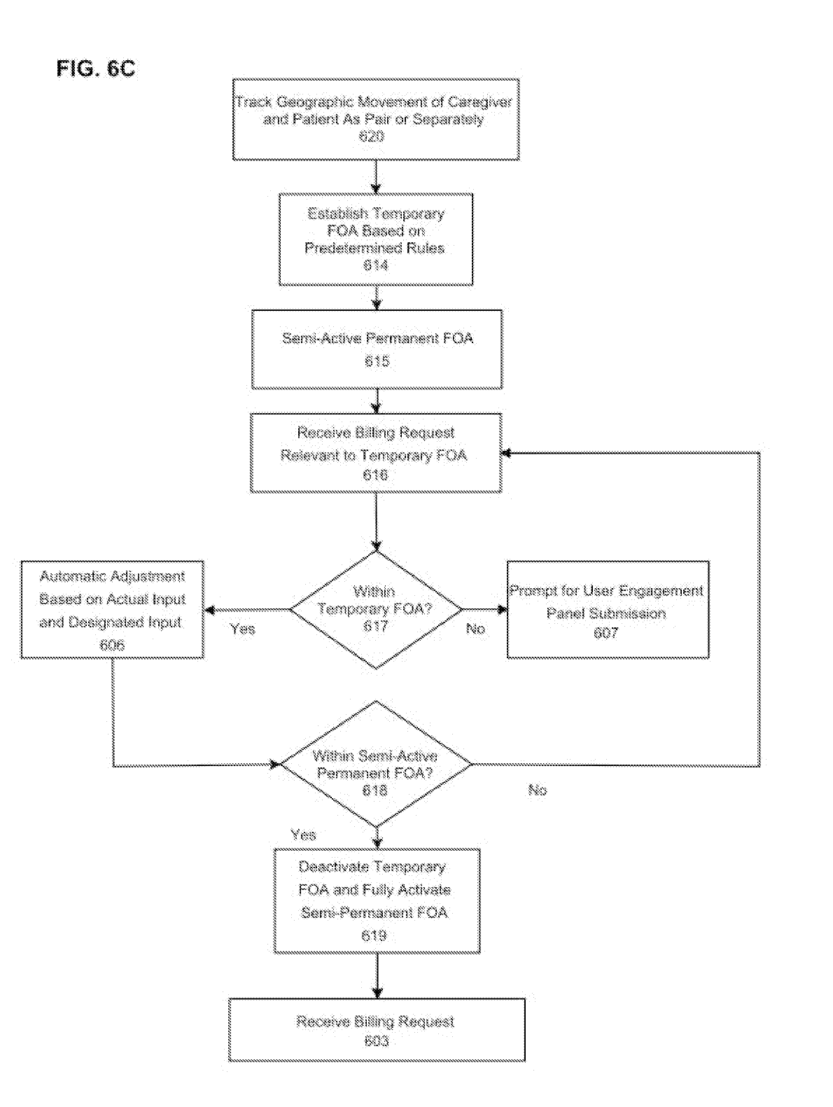

A caregiver might be notified to make a submission including billing relevant data on a relevant user engagement panel 314, The data that is collected through the user engagement panel submissions from one or more caregivers can be used to dynamically update, correct, and supplement database 108. When billing relevant data is submitted on one or more user engagement panels 314 and receives a predetermined number of positive ratings or is verified according to other predetermined rules, it may be used in real-time to update database 108. In turn, an update to database 108 may cause an update to one or more fields of acceptability where relevant. In this manner, a field of acceptability may be dynamically updated and incorporated into database 108 correspondingly. Based on one or more user engagement panel submissions, it may be determined in part whether a field of acceptability is accurate, and whether it should be kept active, deactivated, or incorporated as a semi-active field of acceptability, as further discussed below with respect to FIGS. 6A-6C & 8.

Any user with firsthand experience of the billing relevant information, geolocation, or time, whether it be a service provider, caregiver, vendor, patient, or another individual registered within the system, may contribute to the data of user engagement panel 314. Firsthand experience may be determined at least by geolocation tracking data, where firsthand experience is determined by a user being indicated as physically proximate to a relevant location. A relevant location in some instances may be an actual geolocation under question in a billing request. Proximity may be defined, in an example, as less than 50 feet from a location, or it may be a different definition based on predetermined rules such as 30 or 40 feet. If the user is within a radius based on geolocation data, he/she is indicated to have firsthand experience and is thus qualified to at least rate information on the user engagement panel. In exemplary embodiments of the inventive disclosure, proximity determinations might not be based on a radius; they may be based on being on the same city block or street or based on a geo-fence. These may be predetermined or established at any time. The crowdsourced data or information may or may not be made generally available to other users of the system. If any crowdsourced data or information is identified to be helpful, either through informing other users or by receiving a predetermined number of positive ratings, then a reward may be given to the user who submitted it. This may provide an incentive for users to use and make submissions on user engagement panel 314. It is to be understood by one of ordinary skill in the art that system features may be disposed on other implements not shown or defined by this or other schematic diagrams.

User engagement panel 314 may be organized in one or more different ways. One or more user engagement panels may be utilized, each having its own data storage means. One or more databases may store corresponding information for all relevant data for one or more locations related to one or more service requests. In addition, there may be a central user engagement panel with one or more subsections which apply to a single field of acceptability or multiple fields of acceptability at once.

It will be appreciated that computing system 100 can integrate different functionalities specific to various industries, including the NEMT industry. It is a conventional practice in the NEMT industry to receive service requests from brokers who request that services be provided at very specific times between two locations for which addresses are specified. These specific stipulations are understandably meant to combat fraud and make sure that service requests are completed properly. However, for caregivers in this industry, adhering to the exact timing and addresses specified is not always easy. Unexpected traffic congestion or construction prevent caregivers from arriving on time necessitating billing adjustments. The systems and methods disclosed herein may also be used in conjunction with the systems and methods disclosed in U.S. patent application Ser. No. 15/474,685, filed Mar. 30, 2017, entitled “System and Method for Geo-Aware Transportation Billing Verification,” the entire disclosure of which is incorporated herein by reference. The bona fide nature of these billing adjustments can be confirmed by tracking a caregiver's geolocation using location identifier 304 and assigning a timestamp at the time the caregiver arrives at the patient's home and begins service or picks up the patient or at the time the caregiver leaves the patient's home or drops off the patient to make sure that the timing and/or location based attributes of services provided are within a predetermined field of acceptability. If a service request falls outside of such predetermined field of acceptability, then an alert may be sent to the caregiver and/or an inquiry may be opened to investigate the reasons for such deviation. In this manner, billing clerks can save valuable time, caregivers do not have to risk being fined, and coordinators know that the service requests they booked were completed in good faith.

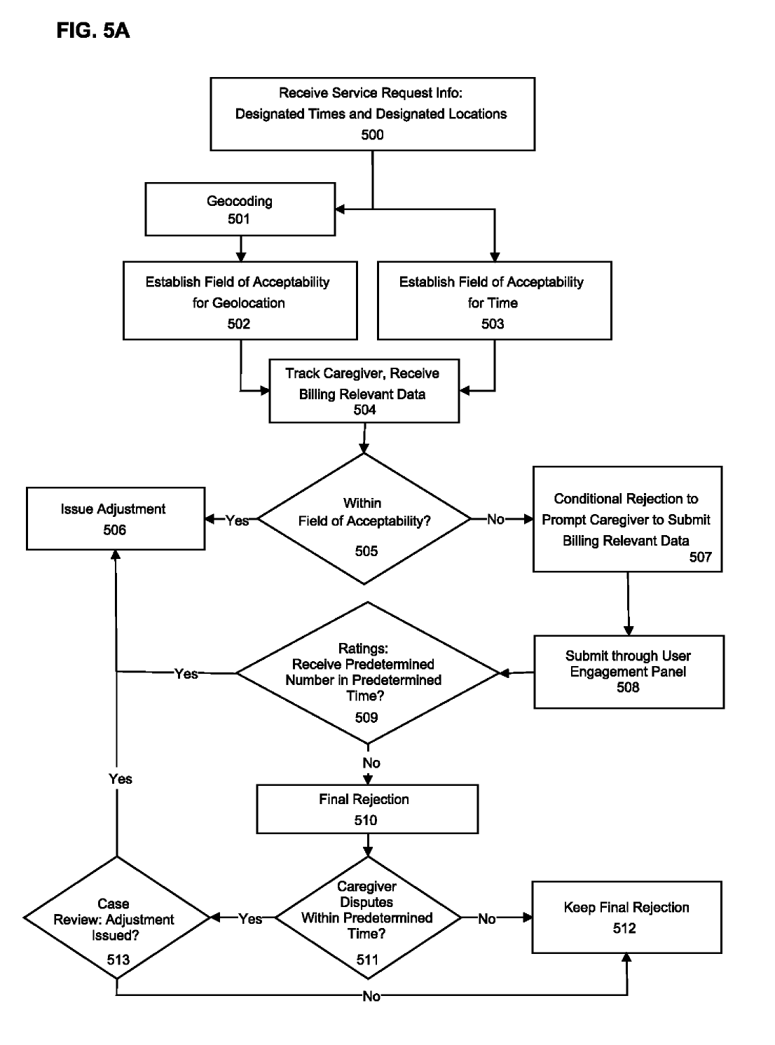

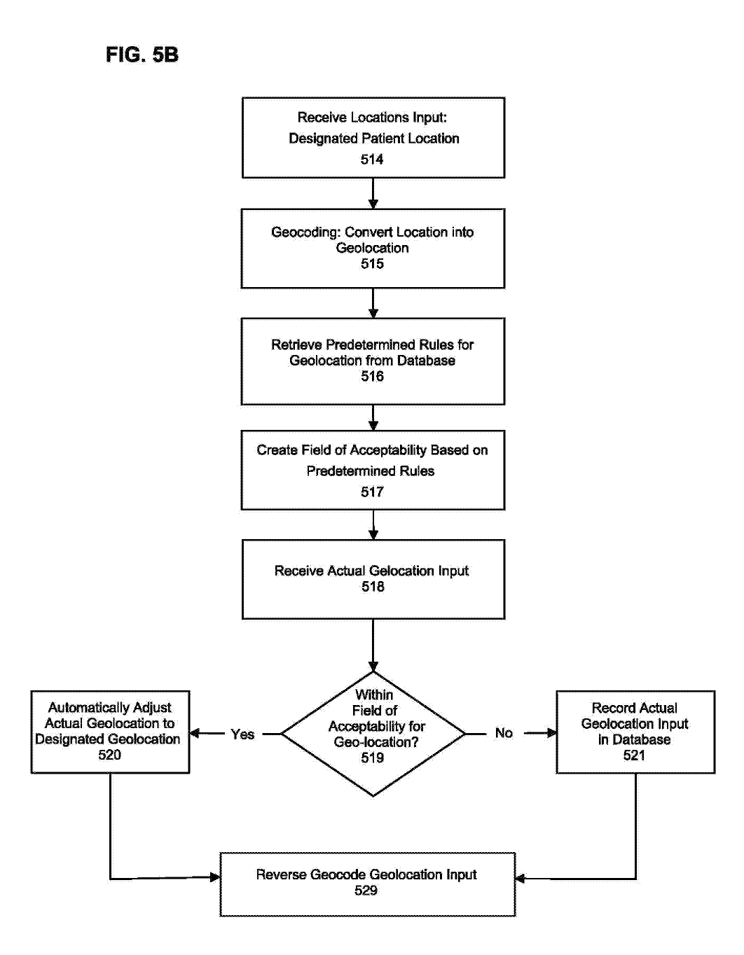

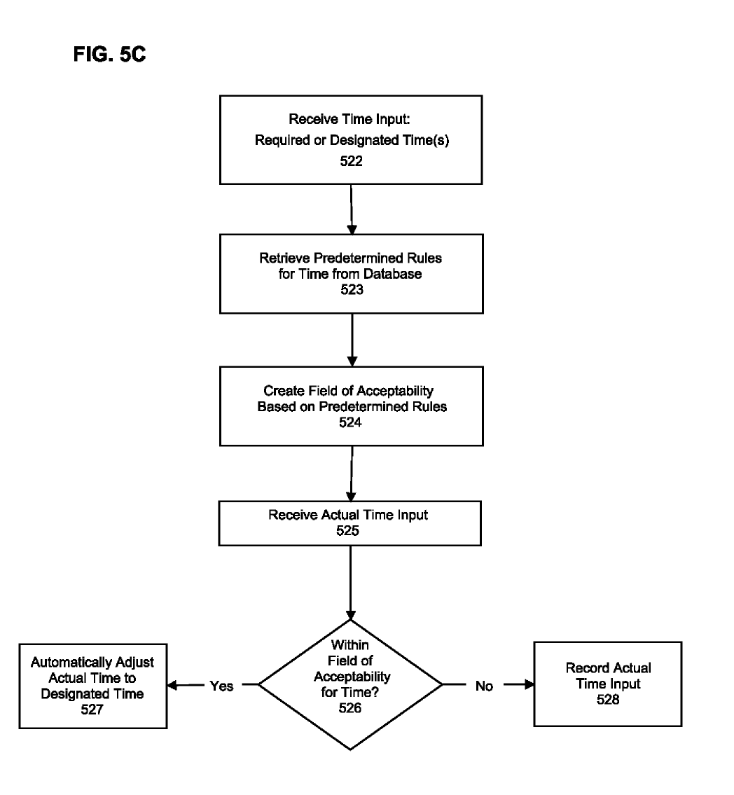

Referring now to FIG. 5A, shown is a flowchart illustrating an exemplary workflow for making a billing request adjustment in the provision of service in accordance with an exemplary embodiment of the inventive disclosure. The first step in the process is to receive the service request information (Step 500), which may include designated locations and designated times related to the service request. The service request information may also include vendor information such as certain field of acceptability stipulations individual vendors (e.g., agencies) may have in accordance with insurance company standards/authorizations. The GIS or one or more servers may implement geoprocessing (Step 501), including geocoding location input as an address into a geolocation including, for example, longitude and latitude coordinates. This may be achieved using a third-party geocoding API such as GOOGLE MAPS. Based in part on the information submitted in the service request, one or more servers 100 can then establish a field of acceptability, which may be based on dynamically adjusted predetermined rules, using values inputted or included in the service request. Geocoding in part allows for the field of acceptability for geolocation(s) to be established (Step 502), by turning one or more locations into geolocation(s). In addition, a field of acceptability for time can be established (Step 503) based in part on the service request input. Once one or both or any other relevant field of acceptability has been established and the service request is underway, the system may track the locations of the caregiver and patient over time and may receive billing relevant data (Step 504). In certain embodiments, the caregiver module may transmit billing request information to one or more servers 100 about the location of the caregiver module, through functionalities such as a GPS receiver. Billing relevant data may be transmitted or otherwise received periodically upon the occurrence of an event (e.g., the caregiver and/or patient stopping for a certain amount of time or stopping within a certain distance of a designated location), or upon an information request (e.g., a service provider, vendor, or other system monitor requesting a status update).

The geolocation of a caregiver and a patient may be recorded directly in reference to a map, which may be provided by a third-party API such as GOOGLE MAPS, a mapping and turn-by-turn direction application provided by Google®, a subsidiary of Alphabet Inc., which features street maps that show corresponding addresses and business names, which may be mined for authentication purposes. A map-based geolocation recording may be used to determine whether the caregiver and patient were at an “acceptable” location such as a nearby park, or somewhere which may warrant further review, such as a casino. It may be appreciated by one of ordinary skill in the art that these locations are exemplary locations, not intended to present a list of fully acceptable and unacceptable locations for a caregiver and a patient to be located. Further, it may also be appreciated that these locations are subject to change, as businesses are known to move locations or close. A review at a later time may categorize a location as acceptable even though it was previously deemed unacceptable.

Based on the service request information and billing relevant data, including the actual geolocation and time, and other relevant information such as pricing information and mileage information etc., it can then be determined whether the caregiver is/was within the field of acceptability (Step 505), and the impact this may have on a billing request. Such determinations may occur after the caregiver module automatically relays billing request information to one or more servers. If the tracked information is within the field of acceptability or matches the service request information, then an automatic adjustment may be made, and the billing request may be approved (Step 506). The information about the adjustment, such as what values were changed and when those changes were made, may be recorded in the system. The records may contain a full accounting of these adjustments and other similar information for a service request and a billing request. If a caregiver is not within the field of acceptability, then the reasons for that happening may have to be reviewed, in which case one or more servers may alert the caregiver through one or more messages through the caregiver module that he/she may be out of the field of acceptability.

This determination may be based on information collected from a patient and used in conjunction with the caregiver module information or on its own. When a caregiver is not within one or more fields of acceptability, a conditional rejection or final rejection may ultimately lead to a temporary or permanent cancellation of payment. In this situation, verification may be needed as to whether the service request was carried out. To do so, one or more servers may send a message to a caregiver when a failure to arrive within a field of acceptability occurs. This may be considered a conditional rejection, and the message may prompt the caregiver to provide one or more reasons and photo(s) for failing to be within the field of acceptability (Step 507). The reason may be a description of circumstances which prevented the caregiver from arriving at the designated time or the designated location, a description of why the caregiver went to a new location outside the field of acceptability (e.g., at the request of the patient and/or out of necessity due to, for example, a laundromat closed for construction), and/or a photograph enriched with time and geolocation data as proof. The caregiver may submit this billing relevant data through the user engagement panel (Step 508). A patient may similarly submit his/her own approval/authorization of the new location or service provided/needed in order to help verify the new location or route thereto.

If a billing request is reviewed, a user (e.g., an agency or administrator/coordinator) could review the billing relevant data, including the one or more reasons provided by the caregiver, and decide to approve or deny the caregiver's reasons. Such approval or denial may exemplify at least a portion of a rating process for the caregiver's submission. The reasons provided may be investigated by users, including one or more other caregivers to determine what problem occurred, and whether they were legitimate. The problem may turn out to be an honest mistake, potentially fraudulent, or not determinable. In certain embodiments, the caregiver may collect a signature or other confirmation such as a fingerprint from the patient that the service request was attempted and could not be completed as designated, and the caregiver may take a photograph and upload it with a written explanation of the situation (e.g., “There was a parade route, 5th Avenue blocked from access.”). A patient verification may take place at the beginning of service, during service, or at the end of service, and may be collected on the caregiver module, on a special purpose device, or even a module that is specifically provided for the patient. In certain embodiments, the system may be configured to enable the patient to add a particular caregiver to a favorites list (e.g., if the patient really likes the caregiver and wants to see him/her again) or a block list if the patient does not ever want to see the caregiver again (e.g., to preclude the system from assigning the caregiver to the patient). Similarly, the caregiver may add a patient to his/her favorites list or block list. In certain embodiments, the system may be configured to allow addition of the caregiver and patient to one another's favorites list when both the caregiver and the patient request it, but to use a preferred list when only one of the two request it. For example, if the patient requests that a caregiver be placed on his/her favorites list but the caregiver does not also request that the patient be added to his/her favorites list, then the caregiver may be added to a “preferred” list of the patient.

In certain embodiments, when the billing relevant data is submitted on the user engagement panel, one or more additional users having firsthand experience may rate it. If the billing relevant data receives a predetermined number of ratings within a predetermined time (Step 509), then there may be an adjustment (e.g., payment), and the billing request may be approved (Step 506) because the ratings have proven the billing relevant data to be accurate. The ratings may be positive ratings, negative ratings, ratings of confirmation, or any other rating type that conveys whether the one or more reasons provided are legitimate or accurate. If the information does not reach the predetermined number of ratings within the predetermined time (Step 509), then a final rejection (Step 510) of the billing request may be issued. If the caregiver does not dispute the rejection within a predetermined time (Step 511), then the final rejection may be maintained (Step 512). If the caregiver believes his/her claim is legitimate and so indicates within a predetermined time period (Step 511), then a service provider (e.g., agency, administrator, or coordinator) may open a case review. In a case review, the service provider may review the reason(s), photograph(s), and/or other information the caregiver submitted. The system may provide the service provider with the same information or more detailed information than what the user submitted on the user engagement panel at Step 508. If the service provider (or a vendor in certain exemplary embodiments of the inventive disclosure) does not believe a caregiver's claim is legitimate and does not approve of a billing request (Step 513), then the service provider may keep the rejection final (Step 512). If the service provider decides to approve a billing request (Step 513), then an adjustment is made and the billing request is approved (Step 506). Factors may include parking restrictions, road rules, constructions sites, temporary road blocks, changes in patient desires/needs, temporary and permanent service location closures, new service location options, etc. Sometimes a caregiver may simply not deem it safe for a patient to exit a vehicle, climb stairs at the location, etc., particularly where the caregiver assesses that it may be safer to pull over and have the patient navigate another nearby location (e.g., another clothing store, laundromat, rehabilitation facility, hardware store, etc.). Road closures for special security events, parades, rallies, or protests may also make it impractical or impossible to arrive at the right location. Such circumstances may be disclosed by the caregiver as reasons for any location or time discrepancies.

FIG. 5B shows a flowchart illustrating an exemplary workflow in accordance with an exemplary embodiment of the inventive disclosure in which the steps of adjusting a billing request or data relating thereto in regards to geolocation is shown. One or more servers receive location inputs that may contain the designated visit or patient locations (Step 514), or, in certain embodiments, multiple locations. Each of these locations is geocoded (e.g., the input locations are converted to geolocations) (Step 515). The geolocations may be used to retrieve the predetermined rules associated with those particular geolocations from the database (Step 516) (e.g., a geolocation where the predetermined rules for the field of acceptability are retrieved, and it is determined the field of acceptability must be based on an area within 120 feet of the designated geolocation).

For the field of acceptability assigned to geolocation, the predetermined rules may create the dimensions of a “buffer,” which are what allow a GIS, such as ArcGIS® (e.g., a mapping platform provided by Esri®—a provider of spatial analysis software), and/or one or more servers to create the field of acceptability. The buffer establishes an area based on a center point which includes all points that qualify as acceptable based on the predetermined rules, and using the buffer, the field of acceptability can be created. In an exemplary embodiment of the inventive disclosure, that center point may be moveable or fixed. With spatial analysis software, certain coordinates with certain attributes may be grouped together within a buffer. The buffer may be based on a set geolocation coordinates. The buffer then is configured to contain all coordinates which are within a certain distance of the center point as its parameters. The coordinates associated with the geolocations all meet certain criteria, and therefore may be grouped based on that similarity. In one example, a field of acceptability may be based on the fact that the caregiver is scheduled to be within 25 feet of a patient. The patient may move around while his/her geolocation is tracked, and it may be determined dynamically whether the caregiver is continuously within 25 feet of the patient. Since the center point is not necessarily fixed, the caregiver and the patient may take a walk in the park, and though they might not be close to the patient's home, the caregiver may still be within 25 feet of the patient. As long as the caregiver is within this field of acceptability, and the tracked geolocation is determined to be a qualified geolocation or route, then his/her geolocation may be automatically adjusted to reflect that of a designated geolocation.

The buffer may establish the field of acceptability by establishing an area which includes all points qualifying as acceptable based on the predetermined rules (Step 517). With spatial analysis software, certain coordinates with certain attributes may be grouped together within a buffer, which may be based on a central set of coordinates translated from an input service request address during geoprocessing. The buffer then is configured to contain all coordinates within a certain distance of the center point as its parameters. The coordinates associated with the geolocations all meet certain criteria, and therefore, may be grouped based on that similarity.

Once the field of acceptability has been established, actual geolocations may be received by one or more servers 100 (Step 518). Based on the input, the system can determine (e.g., continuously or periodically) whether the actual geolocation(s) is/are within the field of acceptability (Step 519) and is/are therefore “qualified geolocation(s)”. If so, then the system may make an automatic adjustment for geolocation (Step 520) before, during, or after receipt of a billing request. For example, a field of acceptability may be established based on a predetermined rule which calls for a size of 300 feet, where the center coordinates for the field of acceptability are based on a designated geolocation of 40° 45′23.2″N, 73° 58′35.8″W. If the caregiver were to visit a patient at 40° 45′23.6″N, 73° 58′35.0″W, which is 105 feet away from the designated visit location (e.g., if the caregiver met the patient in a park adjacent a nursing home rather than at the nursing home itself), then the one or more servers may automatically select the designated input for the visit geolocation, which is 40° 45′23.2″N, 73° 58′35.8″W, rather than the actual input. Similarly, in an example where a caregiver is asked by a patient to travel to a different geolocation to perform covered services (e.g., a medical office, a laundromat, a pharmacy, etc.), once the system detects that the caregiver is no longer within the field of acceptability, the caregiver may provide evidence that the new geolocation should be an approved geolocation for the service request. The system may then adjust the geo-location for the new location to the designated geo-location of the service request. In this manner, future trips by the caregiver to the “new” location(s) will automatically be re-designated or adjusted back to the designated geo-location so the system automatically recognizes that the caregiver is not outside the field of acceptability, and prevents discrepancies or unnecessary data processing during generation, analysis, and approval of billing requests. Such automatic adjustments or re-designations reduce the necessary data processing of the system during the tracking and billing aspects of the inventive disclosure.

In other exemplary embodiments, the field of acceptability may be based on a programmed “snap tolerance,” such as in a GIS, where locations may be “snapped” into groups on a map. The determination of whether the geolocation is within the field of acceptability may, in addition to a coordinate-based analysis, be done by determining a distance between two input points, such as feet, meters, or other measurement. In some exemplary embodiments, geolocations can be geocoded back into human-readable addresses (Step 529). If the actual geolocation is not in the field of acceptability, then no automatic adjustment for geolocation is issued, and the actual geolocation input is recorded by system 100 in database 108 (Step 521). Geolocation input may be geo-processed back into a human readable address (Step 529). Thus, the actual geolocation may be geo-processed back into the actual location.

Certain logical functions may be implemented to provide highly specific and tailored services for the technical challenges that must be solved and may be exceedingly complex to carry out particular if-then scenarios, such as Boolean logic functions. For example, a logical rule may be established to be executed that identifies certain parameters for a field of acceptability. In this manner, many complex conditions may be handled. Various language features that accommodate such conditions can be programmed and implemented through programming languages such as Java. Such languages may include assembly languages, hardware description languages, database programming languages, functional programming languages, imperative programming languages, and so on. In some embodiments, computer program instructions can be stored, compiled, or interpreted to run on a computer, a programmable data processor, a heterogeneous combination of processors or processor architectures, and so on.

The field of acceptability may be previously established (e.g., predetermined) for a past visit or may be based on a default setting. The default setting may depend on the metropolitan area, the time of day, the caregiving agency that scheduled the visit, etc. The predetermined rules that define the field of acceptability may also be subject to variation. For example, a field of acceptability based on distance may not need to be based on a particular radius for an entire three hundred and sixty degree (360°) sweep about a center location, and may instead be a region (e.g., horseshoe shaped) of uniform or non-uniform dimensions. In another embodiment, the field of acceptability may be based on a geo-fenced area with multiple sides of varying length, height, or any other dimension or parameter. It will be appreciated by one of ordinary skill in the art that in some embodiments such regions may be set through adjustments to the field of acceptability. These adjustments may then further be made more accurate by dynamic updates to the predetermined rules. Alternatively, the system and method may further comprise establishing a set of service rules that define and/or redefine the field of acceptability, and may include rules that establish a predefined region for the service location, one or more locations not within the predefined region for the service location, the service time, and a time period which includes the service time. Upon identifying compliance with the service request, the system and method may modify the set of service rules to include the location data generated by the first computing device in defining and/or redefining the field of acceptability.

The field of acceptability may have or require different permissions based on a location. For example, a field of acceptability may include a larger area in a densely populated urban setting in a smaller city because taller buildings may cause signal disruption or other technological limitations. Local and nearby Wi-Fi connections may be utilized to aid in geolocation, particularly where GPS signals are inadequate. A GPS error amount may also be calculated, and the error associated with a signal or at a different area may be factored into the GPS output or received input. The error amount may be different from location to location to compensate for interference or quality of signal.

Next, FIG. 5C shows a flowchart illustrating an exemplary workflow in accordance with an exemplary embodiment of the inventive disclosure, where the steps of adjusting a billing request in regards to time are shown. Once a service request has been submitted by a vendor or other relevant party, the required or designated time input is received (Step 522) by one or more servers. Based on the service request, system 100 may determine whether there are predetermined rules for the field of acceptability based on time by retrieving information from database 108 (Step 523). System 100 may reference a unique ID number or another service request identifier that can be used to query the database for the relevant information. Once the designated time/location and the predetermined rules regarding the field of acceptability based on time for a service request are retrieved, the field of acceptability based on those predetermined rules may be established (Step 524). The field of acceptability based on time may cause times to be grouped by attribute, in which case the defined attribute may be based on a predetermined time or other temporal constraint. Next, the service request can be carried out and the actual time input in a billing request can be received from the caregiver module (Step 525).

Based on the actual time received, it can be determined whether the caregiver is within the field of acceptability for time (Step 526). The designated time will be a known time value, used as a point of comparison to the actual time, where the rule may dictate that any time outside of a time window will not be an acceptable or qualified time, and therefore rejected. The rule may dictate what is a qualified time, such as any time input that is within 10 minutes of the designated time. For example, the designated time is 9:15 AM, and a rule dictates that only times within 10 minutes are a qualified time, then one or more servers 100 or other means of processing may accept any time input between 9:05 AM and 9:25 AM. In other exemplary embodiments, the field of acceptability for time may be created by a rule which makes a qualified time 5 minutes before the designated time while simultaneously creating a field of acceptability that is 10 minutes after. This may mean that a qualified time in this exemplary embodiment may be, for a designated time of 9:15 AM, between 9:10 AM and 9:25 AM. A comparison may be made on the basis of a logical function that creates a timeframe, which may be provided and written through a programming language to be carried out by a computing device. If the time input is within the field of acceptability, then an automatic adjustment for time input may be issued (Step 527) and the designated geolocation input may be selected for purposes of processing the billing request. Adjusting a billing request with regard to time may be done programmatically and automatically according to one or more rules or may be adjusted programmatically or manually after review by a service provider. If the time is not a qualified time, then no automatic adjustment for time will be allowed (Step 528), but the actual time input may still be recorded in database 108.

For a field of acceptability based on time, a designated time may be a known time value or period for when and/or for how long a service visit may be scheduled. The designated time may give context to a predetermined rule which defines a “qualified time.” For example, a qualified time input may be within 10 minutes of a designated visit start time of 9:15 AM, and a predetermined rule that allows times within 10 minutes before and after the designated visit start time as qualified times. A comparison may be made on the basis of a logical function that creates a timeframe, which may be provided and written through a programming language to be carried out by a computing device. Accordingly, one or more servers or other means of processing may accept any time input between 9:05 AM and 9:25 AM. Any time input between this 10-minute range is considered a qualified time as it is within the field of acceptability. The actual time may be adjusted to the designated time. If a caregiver arrives at 9:17 AM, the time input may be adjusted to 9:15 AM and/or recorded as 9:17 AM but considered a qualified time. Adjusting actual time to designated time may be done programmatically according to a rule or may be adjusted programmatically or manually after review by an agency. If the time is not a qualified time, it will not be adjusted automatically. Instead, the actual time input will be recorded, and the caregiver may provide reasons for the discrepancy, as described in more detail below.

The input information may be further encoded using another key by a third-party such as an insurance company. This may allow the data to be shared between the third-party and the agency without worries that any of the data will be altered in bad faith. For example, an insurance company may provide an additional encryption layer to data forwarded thereto and therefrom. The agency can then ascertain that the information sent is from a legitimate third-party by verifying the credentials found in the further-encrypted key. The agency can then return data with or without this key which contains the time and location information requested for billing.

It will be understood that FIGS. 5B-5C may be instantiated as subroutines which are intended to describe more specific steps regarding establishing the fields of acceptability as mentioned in FIG. 5A, rather than figures that establish the only process by which one or more fields of acceptability may be established. The field of acceptability may relate to the caregiver traveling a certain total distance, traveling along a certain route, travelling to specific visit locations or other authorized locations, staying with or near the patient, obeying traffic rules, adherence to service requirements, etc. The predetermined rules may also be subject to change based on collected data, which in some exemplary embodiments may be analyzed by artificial intelligence (AI). AI may determine patterns which may subject the field of acceptability to change in certain conditions. For example, weather conditions may affect the ability of caregivers to timely arrive at some locations, take longer to run errands for the patient, and/or increase traffic congestion. In such situations, AI may change the field of acceptability based on predetermined rules.

It will to be understood by one of ordinary skill in the art that an approach to changing the field of acceptability based on data collection may be applied to numerous other factors not limited to weather conditions. Furthermore, the field of acceptability based on distance does not need to be based on a certain radius. However, to qualify for payment of a billing request, the caregiver must meet all or a certain number of variables. For example, to qualify for payment, a caregiver must provide service as requested, or otherwise have the billing request variables adjusted-whether through automatic adjustment, adjustment through the user engagement panel, or adjustments made following a dispute. However, cases may arise where an adjustment is not made to such one or more variables even though the caregiver ultimately provided the services of the service request. For example, if a caregiver does not have legitimate reasons for picking up or visiting a patient at a location different from the designated pickup or visit location, but still visits with the patient and/or provides service within other correct or designated time periods, then the one or more reasons for the caregiver not making a pickup at the correct location may be collected and analyzed. However, as the service request was satisfied and a billing request was submitted, the caregiver may be paid (e.g., in full, in part, or not at all) as there are numerous situations in determining payment that may be best carried out at the discretion of a service provider, one or more additional users, a vendor, or the system. In other exemplary embodiments, paying a caregiver the correct amount for a billing request in some situations might not be based on a discretionary determination.

Situations may arise in which a caregiver or a patient does not participate as expected, or where one party is unable to locate the other (e.g., a no-show or an attempt to meet at a crowded location). If the caregiver does not show up, then no service has been provided and no billing requests should be generated. If the patient does not show up, then there may be no verification from the patient. There may also be situations when the caregiver or patient cancels the service request before or during the service. Programmed work-arounds may be utilized for such situations and may be verified. One such verification factor may include tracking geolocation and time information and determining whether the caregiver is within a field of acceptability. For example, meeting a verification factor requirement may involve a caregiver arriving at a certain geolocation and staying there for a certain duration of time, such as 10 or 15 minutes or another predetermined time. If the caregiver is unable to find parking during that time due to a parking regulation, he/she may circle the block or wait at another nearby location, and this may be considered wait time if appropriate. Another verification factor may relate to an effort to contact another party. A text entry field may be provided on a mobile device in order for the caregiver or patient to register information regarding the other party not showing up, and such information may be submitted through the user engagement panel or included in a billing request.

A no-show or similar situation might or might not be related to the field of acceptability. However, the patient module, the caregiver module, the service provider module, or the vendor module may implement features to account for numerous situations which may arise, such as the service provider entering certain clarifying information, etc. If the patient does not require care, a caregiver can still submit a billing request manually or automatically (e.g., at expiration of a timer programmed to start upon arrival.

Turning next to FIG. 6A, depicted is a flowchart illustrating an approach for establishing and updating the field of acceptability in accordance with exemplary embodiments of the inventive disclosure. The party or entity which bears responsibility for paying for provided services (e.g., an agency, insurance company, etc.) may have authority to establish the initial (preliminary/temporary) field of acceptability and authorize temporary or permanent updates thereto. The method of establishing the field of acceptability may include different stages during which the parameters of the field of acceptability differ. In certain embodiments, three “types” of fields of acceptability may apply. The field of acceptability may first be established as a preliminary field of acceptability based on a vendor's or agent's discretion or common sense, i.e. arbitrarily, or otherwise based on historical data and/or an individual insurance company's mandates, guidelines, and rules for service, if available. A preliminary field of acceptability may thus be established based on default parameters, automatically or manually (Step 600). Such default parameters may be predefined by a vendor, a vendor's agent, or another relevant party, agency, or insurance company. Default parameters may be arbitrary but updated in response to how caregiver services are provided. System 100 may employ significant data collection (Step 601), during a predetermined number of service requests associated with a same patient, and/or with a same location or a plurality of locations close to one another. During this phase, user engagement panel 314 may be employed to collect information or data which may help in eventually transforming the preliminary field of acceptability to a more accurate permanent field of acceptability. Data collection may help establish a more accurate permanent field of acceptability, and the collection process may take place for any length of time, as necessary. Data collected may be stored in database 108. However, “permanent” as used herein with regard to the field of acceptability does not indicate that the field cannot or will not be changed. The term “permanent” is used herein with respect to “field of acceptability” to indicate that it may be applicable over a longer period of time. There may be circumstances where the permanent field of acceptability is subject to change based on collected data or reset, such as a permanent street closure, a change in rules or regulations, or by the caregiver and patient travelling to new locations which the patient (and ultimately the agency) approve.

The default parameters of the preliminary field of acceptability may be generally based on services provided previously until they are updated to reflect the services more clearly. Updates to the default parameters may cause the field of acceptability to expand or to shrink for improved accuracy. If certain circumstances call for a field of acceptability to be reset, then an additional data collection period may occur again. In certain embodiments, the data gathered during this data collection period may change a preliminary field of acceptability to a permanent field of acceptability.

Sometimes it is impossible for a caregiver to be within the same vicinity as the patient. Unless the caregiver's scheduled tasks were to assist the patient with bathroom and shower needs, it would be reasonable during visit times for the patient to prefer to be in the bathroom alone for privacy. Patient and caregiver tracking may thus be configured to accommodate this concern, and the field of acceptability may similarly allow flexibility for such separation between the remote computing devices of the patient and caregiver, which preferably remain on their persons or very closely near them during the entire time of the caregiver visit. As tracking may require the caregiver to “clock in” at certain time intervals, the health care agency may provide a grace period for the input intervals (e.g., a set number of minutes before and after each prompt is communicated to the caregiver). During this period, if the caregiver and patient both input their information, the caregiver will be deemed compliant with the scheduled visit time.

The home health agency may employ a system with more than one server in a distributed structure with support from data centers that may be located anywhere around the globe. These implementations may be communicatively linked and cross platformed so an administrator on a computing device is provided with information relevant to each caregiver visit. Various features of the system can be implemented through computing devices that allow method steps to be processed and outputted by one or more servers. Server-side architecture may be implemented through a software program configured to coordinate communication between a module and the backend systems that allow for bringing up data and image processing, as well as storage and some calculation features. In certain embodiments, billing relevant data, which is also be referred to herein as service relevant data, is collected continuously or periodically from one or more computing devices. The system and method may further include aggregating the billing relevant data received from the plurality of computing devices located within a predetermined distance from the service location and storing the aggregated billing relevant data. The field of acceptability, the predetermined rules, and/or a set of service rules may be updated dynamically in accordance with this aggregated billing relevant data. The aggregated billing relevant data may include GPS data corresponding to one or more geolocations and time data corresponding to one or more times or days of transmission of the aggregated billing relevant data. For example, the system may receive aggregate data for a plurality of service requests for a patient at a central location (e.g., a home serviced by one or more caregivers), and used to adjust a field of acceptability around the central location (e.g., a pre-set distance may be too large or too small for the location). If location data associated with the caregivers reveals that caregivers stay within 50 feet of a location (e.g., the home) 90% of the time, then the predetermined rule for establishing the service field about the home could be 50 feet. However, if 90% of caregivers complete service while staying within 20 feet, then the acceptable service field may be automatically reduced after completion of a preset number of service requests. Other percentages, such as 50%, 75%, 80%, etc. may be utilized as the threshold for changing adjusting the field, and other distances (e.g., 20 feet, 25 feet, 30 feet, 40 feet, a block, a mile, etc.) may be utilized as a distance to set from the central location.

As discussed herein, the field of acceptability can take any number of forms, can encompass many locations and routes, and is not limited to a radius, circle, or any other particular geometric shape. In this manner, aggregate data may be used to update the service field. If the number of adjusted billing requests supported by the same type of legitimate reasons reaches a predetermined number, then the preliminary field of acceptability may be deactivated and transformed to a permanent field of acceptability. Generally, if the data collection shows caregivers repeatedly outside the preliminary field of acceptability, then the preliminary field of acceptability may be deactivated, modified, and/or transformed into a larger permanent field of acceptability based on relevant data collected. If, on the other hand, the data collection shows that caregivers repeatedly are most often closer to the designated location than the maximum distance established by the preliminary field of acceptability, then the preliminary field of acceptability may be transformed or modified into a smaller permanent field of acceptability. In this manner, based on data gathered during data collection, the permanent field of acceptability is established (Step 602), and represents a more accurate reflection of when or where caregivers are providing services. Since real-life situations often change and may affect the location(s) where services are provided, the field of acceptability may need to be repeatedly updated.

Preferably, only one maximum field of acceptability exists at any given time but can apply to an array of locations. The status of the field changes according to patient needs in conjunction with the types of services the caregiver provides. Each field of acceptability contains three characteristics, namely, fully-active, semi-active, and non-active. System 100 may employ continuous data collection to maintain the accuracy of each field characteristic. Additionally, continuous data collection can aid the home healthcare agency administration to keep track of particular caregiver and patient travel patterns.

In certain embodiments, the preliminary field of acceptability may be based on a caregiver's initial visit or by the home health agency administration. The preliminary field set-up may utilize a coordinator approach and/or previous visit (historical) data. An agency administrator may initially decide what area range (e.g., region) outside the patient's home is reasonable. Eventually, this range dictates initialization of the preliminary field automatically by the system during subsequent visits. Alternatively, system may automatically generate the preliminary field based on pre-set distances and ranges for a given location within a given geographic area. Previous visit (historic) data may include stored histories for a plurality of caregivers proximate to that location and assigning a reasonable range.

In certain embodiments, after sufficient data is collected from a service visit, the data is sent to an administrator for analysis and review. Upon review completion, the administrator decides whether the data is reasonably sufficient to deem the location a permanent field of acceptability. Occasionally, exigent circumstances such as emergencies or any other necessities may arise which require a caregiver to travel outside of the permanent field of acceptability. Such circumstances may temporarily change the field of acceptability for a limited time.

In certain embodiments, system 100 receives a billing request (Step 603) and determines whether the caregiver was within the field of acceptability (Step 604). If the caregiver was not within the field of acceptability (or was not for a requisite period of time), a conditional rejection may be issued, and the caregiver may be prompted to submit billing relevant data in the form of one or more reasons or photographs through a user-engagement panel identifying that services were in fact provided to the patient at the scheduled time (Step 605). As disclosed with respect to FIG. 5A, a billing request submitted by or on behalf of a caregiver may be subject to a full or conditional rejection, depending on the circumstances. Once submitted through user engagement panel 314, billing relevant data submitted by the caregiver is reviewed and verified. If an adjustment is made to the billing request or if the caregiver's billing request is rejected, the caregiver may still have a chance to dispute the rejection within a predetermined time. It will be appreciated that if the caregiver submits a billing request as soon as he/she completes one or more services, he/she may be given notice by an alert over his/her mobile device that he/she is deemed outside the field of acceptability, and be given an opportunity in real-time to collect and input evidence (e.g., pictures of a location, a signature and timestamp authorization from the patient, any reasons which are fresh in his/her mind). Alternatively, if the billing request is submitted long after services are provided (e.g., a month later) on behalf of the caregiver, then the caregiver may not have evidence or remember the particular service. Thus, caregivers are incentivized to journal or otherwise record any reasons why he/she might be outside the scope of the field of acceptability, and to collect patient authorization at the time. In certain embodiments, the caregiver may be given the option to upload such evidence even in the absence of a real-time alert or billing rejection so that the information is already in the system at the time a billing request is sent. In yet other embodiments, if the caregiver is unaware that he/she is outside of the field of acceptability, and the billing request is submitted on his/her behalf days or weeks later, then time sheets and/or patient certifications/authorizations may be utilized to prove compliance with the patient's service request.

If the caregiver is determined to have been within the field of acceptability, then an adjustment may be made to the billing request based on the actual input and the designated input (Step 606). Based on predetermined rules, the system may determine whether the data collected (from one or multiple caregivers) warrants an update to the permanent field of acceptability (Step 607). Such determinations may be accomplished by evaluating actual inputs (e.g., actual geolocations or actual times) compared to designated ones and maximum field allowances. By way of example, if for a designated geolocation, the field of acceptability accepts an input within 100 feet, then 100 feet is deemed the “maximum allowable input.” If the provision of service repeatedly occurs at only 25 feet from the designated location, then this may be considered evidence that the field of acceptability is too permissive. If the actual inputs are not substantially different, then the data collected would not warrant an update to the permanent field of acceptability (Step 608). If the data collected does warrant an update to the permanent field of acceptability, then this may cause such an update based on predetermined rules (Step 609). The update may tailor the field of acceptability to the relevant size based on the billing relevant data (e.g., the field of acceptability may be made smaller or expanded to be more permissive). If a caregiver is not within the field of acceptability, then he/she may be prompted to submit the reason(s) on user engagement panel 314. If the caregiver is within the field of acceptability, then his/her visit location may be automatically adjusted, and data may be collected from the visit.

As discussed above, if a predetermined number of caregivers provide the same or similar reasons through the user engagement panel, then a temporary field of acceptability may also become a permanent field of acceptability similar to the process of a preliminary to permanent field. This determination may also be based on how long the temporary field of acceptability has been in place. It will be understood that the terms preliminary, temporary, and permanent as used herein are terms intended to explain different stages of establishing and updating a field of acceptability, and that these terms are not intended to be the only definitions or descriptions of the ideas they convey. In certain embodiments, system 100 may also periodically issue an alert to the caregiver to notify him/her of being within the field of acceptability so the caregiver does not need to guess or worry about future billing issues. The caregiver preferably receives an alert or alerts notifying him/her of being outside the field of acceptability as discussed above. Such alert(s) may indicate that the caregiver needs to proceed closer to a designated location in order to be authorized, or to submit evidence/authentications of new locations or routes.

Shown in FIG. 6B is a flowchart illustrating an approach for establishing a temporary field of acceptability in situations where, for example, a caregiver is asked by the patient to drive him/her to a new location outside the permanent field of acceptability (e.g., travel to the pharmacy, a laundromat, hospital, supermarket, etc. not already within the permanent field). Accordingly, the caregiver may be prompted to submit billing relevant data (Step 605) so that the system can determine if adjustments have or need to be made (Step 610). If a predetermined number of adjustments has not been reached, then the submissions and other relevant billing request information are recorded in database 108 (Step 611). If an adjustment is ultimately issued, then system 100 may next determine whether a predetermined number of substantially similar adjustments have been made for the same patient (e.g., based on the location, based on the reason, etc.) (Step 612). If a predetermined number of adjustments have been made, then system 100 may analyze the reason(s) for the adjustment and whether the reason(s) is/are long-term ones. If the reason is determined to be long-term, then the temporary field of acceptability may be updated based on predetermined rules (Step 613). If the reason is not long term, then the temporary field of acceptability may be established based on predetermined rules (Step 614).

In certain embodiments, the temporary field of acceptability may be a semi-active field (Step 615), and be used to accommodate emergency situations which may be proven later by a caregiver's explanation for either not arriving to or travelling outside of the permanent field of acceptability. The caregiver may be given a timeframe and a request for an explanation through the user engagement panel as to why the caregiver and one or more patients are outside of the established permanent field of acceptability. If the reason provided by the caregiver is valid, then a temporary field of acceptability is created for a limited time duration.

According to an exemplary embodiment of the present invention, certain fields of acceptability may be in a state of activity such as active, inactive, or semi-active. An “active” field of acceptability herein is intended to refer to a field of acceptability that is currently applicable and applying to time or location in a billing request. An “inactive” field of acceptability may refer to a field of acceptability that has been deactivated because it is considered inaccurate. “Semi-active” may refer to a permanent field of acceptability that may be incorporated with a temporary field of acceptability, and fully activated to the permanent field when the temporary field is deactivated according to predetermined rules. Certain conditions may trigger full activation of the semi-active permanent field of acceptability as disclosed in FIG. 6C.

FIG. 6C is a flowchart illustrating an approach for updating a temporary field of acceptability in accordance with exemplary embodiments of the inventive disclosure. When a temporary field of acceptability is established and in place (Step 614), which may also or alternatively be a semi-active field (Step 615), a caregiver may submit a billing request relevant to the established temporary field (Step 616). System 100 determines whether the caregiver was within the temporary field for a requisite period of time (Step 617). If the caregiver was within the temporary field, then system 100 may make an adjustment to a billing request based on an actual input and a designated input (Step 606). If the caregiver was not within the temporary field, then he/she is prompted to submit billing relevant data via user engagement panel 314 (Step 607) in support of an adjustment.

In certain embodiments, a series of conditions may cause the temporary field of acceptability, once established, to be deactivated. When a caregiver is determined to have been within a temporary field of acceptability and an adjustment is made (Step 606), system 100 may further determine whether the caregiver was within a semi-active permanent field of acceptability (Step 618). If the caregiver was within the semi-active permanent field of acceptability, this may be an indication that the temporary field of acceptability no longer applies. Such a scenario may trigger deactivation of the temporary field of acceptability and full activation of the semi-active permanent field of acceptability (Step 619). For example, the temporary field of acceptability was established because a caregiver was asked by a patient to drive him/her to, for example, the pharmacy, which may be within the semi-active permanent field of acceptability. If the caregiver was not within the semi-active permanent field of acceptability, yet was still within the temporary field of acceptability, the temporary field of acceptability may still apply.

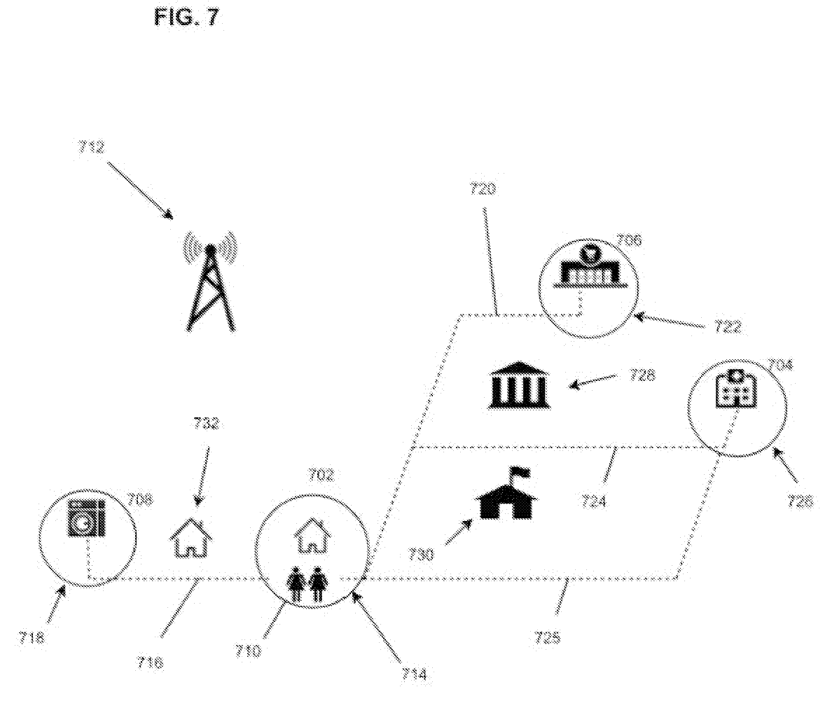

Turning next to FIG. 7, shown is a diagram illustrating the application of the field of acceptability based on distance in accordance with exemplary embodiments of the inventive disclosure, in which home healthcare services are tracked accurately and conveniently. In particular, caregiver/patient 710, shown at patient home 702, may have computing device 128, which may be a mobile telephone or smartphone, specifically configured to track services provided by the caregiver. Various other establishments may be located within the vicinity of the patient's home 702, such as laundromat 708, hospital or medical facility 704, supermarket 706, museum 728, pub 730, restaurant 732, etc. As part of the care to be provided in accordance with the service request, the caregiver may be responsible for traveling to any one of laundromat 708, hospital or medical facility 704, or supermarket 706, either with or without the patient, during the time frame they care for the patient at the patient's home 702. Conversely, certain other establishments may be off-limits or otherwise not authorized for the caregiver to visit during the time he/she should be caring for the patient. Also, communication tower 712 is preferably in sufficient proximity to permit communication between system 100 and one or more computing devices 128. Computing devices 128 may be used to verify that the caregiver has worked with the patient within the parameters of the service request, including verifying that the caregiver did not spend time outside of the required locations or routes thereto when rendering the services during the time frame(s) designated in the service request.

In certain embodiments, the patient and caregiver are registered with system 100, and the patient's address and schedule for care is stored in database 108. A caregiver can then be assigned to provide care for the patient in accordance with, for example, service requests outlining the assigned appointments during which they are to care for the patient. Caregiver module 434 may interface with computing device 128 to add appointments to the caregiver's computing device 128. When the caregiver arrives at patient home 702 pursuant to an assigned service request, the computing device 128 may transmit a signal to system 100 indicating that the caregiver is at patient home 702 to begin rendering services. The system may periodically ping the location of computing device 128 to identify and record its location, for example, every few minutes until the caregiver manually indicates completion of the services, or if the detected location data indicates that the caregiver moves outside of the field of acceptability (i.e., by traveling away from patient home 702 or away from other designated locations shown in FIG. 7 or routes thereto) or is at a time that is outside the time period for rendering such services.

Additionally, a vehicle (which may include a caregiver module) may be driven by a caregiver providing services pursuant to a service request with a primary designated location as the patient's home 702. As established by certain predetermined rules and/or the service request, one or more fields of acceptability may be established for the caregiver to render services. For example, in one embodiment, a field of acceptability 714 may be established for the caregiver to render services to the patient at patient home 702 only. However, the caregiver may additionally be responsible for taking care of the patient's laundry, and thus must travel to laundromat 708 on occasion. Alternatively, the caregiver may have to take the patient to appointments at hospital or medical facility 704 on occasion. Accordingly, temporary fields of acceptability 718/726 including routes thereto from the patient's home 702 may be established (e.g., at laundromat 708, hospital 704, and/or routes thereto). If these locations are later confirmed as verified services to be provided pursuant to the service request (and verified locations), then temporary fields of acceptability 718/726 may be incorporated into or included in the field of acceptability 714 as a permanent field of acceptability. It will be appreciated that there may be more than one feasible route or path from one service location to another. For example, a caregiver may travel along path or route 724 or 725 to take the patient to a hospital or medical facility 704. In certain embodiments, each of the routes 724/725 may be automatically included as part of temporary field of acceptability 726. Additionally, paths or routes 716/724/725 over which the caregiver must travel will also be incorporated into the temporary field(s) of acceptability for the particular service request. Of course, the temporary field of acceptability will include at least both the time and location points of routes 716/724/725 from patient home 702 to laundromat 708 or hospital 704, respectively. So long as the caregiver does not deviate from the time and location parameters of the routes to laundromat 708 and/or hospital 704, system 100 will consider the caregiver to still be within the field of acceptability.

In certain embodiments, if a caregiver traverses a new route between two approved locations which are already part of the field of acceptability, and the caregiver traverses the new route within a pre-set amount of time, then the system may automatically make/deem the new route part of the field of acceptability without any user input or administrator, patient, or agency approval. In such circumstances, there may be no need for establishing a temporary service field for the route because the two service locations to which the route connects have already been approved, and the caregiver was able to traverse the new route within a reasonable time. It will be appreciated that the field of acceptability may be tailored to specific pathways along routes which caregivers travel, and that unapproved and/or rejected regions or locations may be bounded or circumscribed by approved routes or locations. For example, an unapproved location may fall between two or more approved locations/regions. Additionally, the field of acceptability around a particular location at an end of a route may be increased or decreased in accordance with aggregate data as described above.

In an exemplary embodiment of the inventive disclosure, system 100 may enable verification of billing information associated with caregivers by automatically adjusting the location data or the time data generated by the first computing device to an approved time and location of the service request. In this manner, when the service request is billed, the system 100 does not need to compare data from multiple locations, thus reducing processing time. Additionally, if a caregiver is again detected to be at the same location during a time when he/she is caring for the same patient, system 100 automatically recognizes the location as an approved location for the service request.

Continuing with respect to FIG. 7, the first time the caregiver travels to laundromat 708, system 100 may issue a notification to the caregiver that he/she is not within the field of acceptability for the patient. Upon receipt of evidence (i.e., from the caregiver, the patient, or otherwise) that the caregiver must travel to laundromat 708 as part of the services being rendered to the patient, system 100 then designates or adjusts the location data for laundromat 708 (and all location data point along route or path 716) to match or be the same as the location data designated in the service request (e.g., patient's home 702). In this manner, all future times the caregiver travels to laundromat 708 during a time frame he/she is caring for this particular patient, system 100 recognizes laundromat 708 as having the same location data as patient's home 702 and recognizes the caregiver as being within the field of acceptability. Alternatively, the rules defining the field of acceptability may be adjusted or otherwise modified to include the location data associated with laundromat 708 (and all location data point along route or path 716) thereby expanding the field of acceptability.

Occasionally, a caregiver may have to make a one-time trip to, for example, supermarket 706, either with or without the patient. In such embodiments, system 100 may detect that the location of the caregiver is outside of field of acceptability 714 as he/she travels along route 720 to supermarket 706. Upon detection of the deviation from field of acceptability 714, system 100 may transmit a signal, alert, or notification to the caregiver informing him/her of the deviation, at which point the caregiver may return to a location within field of acceptability 714 or may submit information or evidence that he/she is deviating from the designated field of acceptability while still performing services pursuant to the service request. System 100 may then again create a temporary field of acceptability 722 which includes route 720 for the service request. Before issuing payment to the caregiver for rendering services to the patient pursuant to the service request, system 100 may require further verification that the caregiver's deviation from field of acceptability 714 was in fact to provide services for the patient pursuant to the service request and authorized by the patient. Other legitimate reasons for a caregiver to travel outside of the designated field of acceptability to render services may arise. Flexibility is provided to patients and caregivers while allowing for better tracking and verification.

In certain embodiments, a biometric unit on computing device 128 may be utilized to ensure that the caregiver has not had another person interact with the device or provide the services to the patient on the caregiver's behalf. For example, the caregiver may be asked to take a picture of themselves, and the photo may be uploaded to a central server so that system administrators may at that time, or at a later time, verify that the actual worker was at the location (and did not, for example, pay someone less money than they were making to sit in for them). In certain implementations, the patient may also be prompted for biometric input or prompted to provide a password when the treatment session has ended, verifying they were at the appointment and that treatment was provided satisfactorily. Once the caregiver has completed the appointment, they may indicate such completion to the application, such as by selecting an icon that has been visually displayed on computing device 128. Such an action may simply stop the clock from running, or may result in other actions occurring, such as in computing device 128 reporting to a central server that the appointment has been completed, and then receiving back from the central server new instructions for the caregiver.

The tracking and communications module may allow for tracking information transfer in multiple directions, such as from caregiver/patient to agency, agency to caregiver/patient, caregiver to patient, patient to caregiver, etc. Thus, this module may have a bi-lateral communication function. This bi-lateral transfer of data ensures that the caregiver is always within the proximity of the patient. A record is automatically created when the caregiver enters the appropriate information upon arrival. Newer information may be gathered with each subsequent visit to build a patient record dynamically. Such information may also be gathered at various intervals throughout the day. The patient record can be customized by caregivers according to patient needs. As an example, the visiting caregiver may enter a patient number, alpha numeric data, and/or other data into the mobile application or device via an input interface, such as a keypad, touchscreen, to start the visit. A visit record is then generated and transmitted to the home healthcare agency's administration system over a communication network and alerts the administration or the coordination department that a visit has just started. As information from the previous visits to the patient has been collected, recorded, and updated dynamically, the central server may then automatically respond to the caregiver's mobile device with a specific list of tasks for the caregiver to perform based on this information in the patient record. Alternatively, information and tasks may be manually inputted by the administration or coordination department and sent to the visiting caregiver. The information gathered for these patient records may then be used for monitoring caregiver compliance, administrative needs, and proper billing practices.

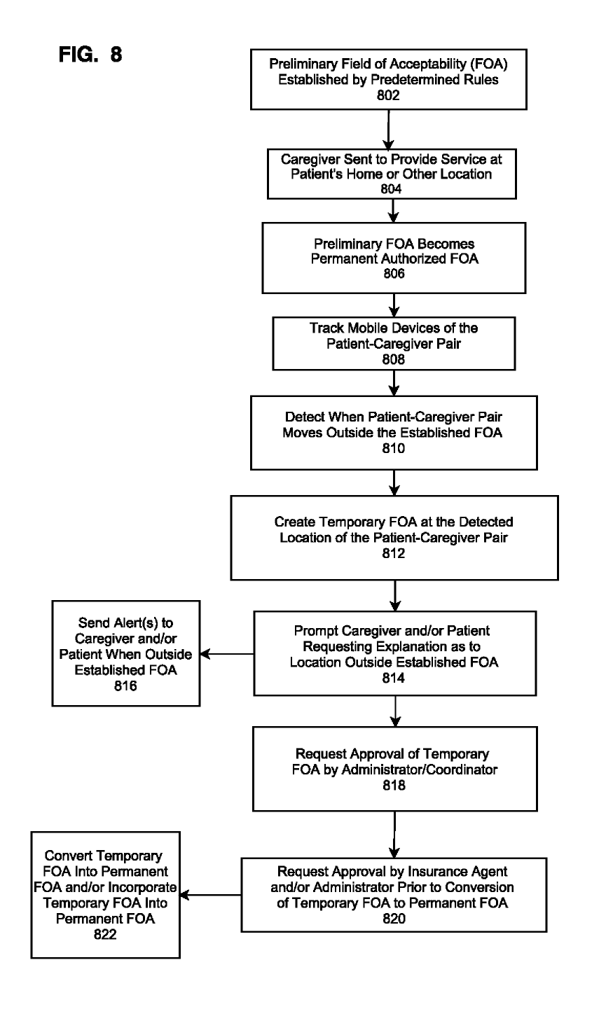

Referring next to FIG. 8, shown is a flowchart illustrating an alternative approach for creating a temporary field of acceptability and updating the temporary field in accordance with exemplary embodiments of the inventive disclosure. A preliminary field of acceptability is initially established by the system in accordance with predetermined rules (Step 802) any time after a service request is received by system 100. At the time indicated in the service request, a caregiver is dispatched to arrive at the patient's home or place where services are to be provided (Step 804). In one embodiment, once the caregiver arrives at the patient's home or at another specified location for performing services, the preliminary field of acceptability may be converted to a permanent field of acceptability upon confirmation from the patient (e.g., through patient module 436), or from the combination of patient and caregiver (Step 806). The system may be configured to then track the mobile devices 128 of the caregiver-patient pair (Step 808), such that it detects if the pair moves outside of the established permanent field of acceptability (e.g., outside of any designated locations or routes such as those described with respect to FIG. 7) (Step 810). Upon detection of movement outside of the established field of acceptability, a temporary field of acceptability may be created at new location(s) where the pair traveled (Step 812), as well as the routes traveled to get there. The patient and/or caregiver may then be prompted (e.g., through a user engagement panel on their computing device) to provide an explanation and/or evidence as to the new location and whether or not it is within the scope of the services scheduled to be provided by the caregiver (Step 814).

For example, the caregiver may take a picture of the laundromat, and the picture may have metadata evidencing the location of the laundromat, as well as the time the caregiver arrived there (e.g., with or without the patient). Alert(s) may additionally or alternatively be sent to the caregiver when he/she moves outside of the established preliminary field of acceptability (Step 816). The caregiver may then be given the option to return to a location within the established field of acceptability or provide an explanation and/or evidence that he/she is still working within the scope of the service request so that the field of acceptability can be adjusted. If it is determined that the caregiver was within an acceptable field according to the service request or services to be provided, an adjustment to the field of acceptability may be made upon approval by an administrator and/or coordinator (Step 818). The system may also require that the temporary field of acceptability receive approval by not only the caregiver and patient, but also by the insurance agent and/or the administrator (Step 820). Upon approval, the temporary field of acceptability may be incorporated into or converted to a permanent field of acceptability (Step 822). For example, the temporary field of acceptability was established because a caregiver was asked by a patient to drive him/her to, for example, the pharmacy, which may be within the semi-active permanent field of acceptability. If the caregiver was not within the semi-active permanent field of acceptability, yet was still within the temporary field of acceptability, then the temporary field of acceptability may still apply.

Additionally, an acceptable range beyond any of the fields of acceptability may be temporarily established for either the caregiver or the pair to travel, such as a reasonable amount of feet away from the field of acceptability upon arrival. For example, a field of acceptability may be established on the route from the patient's house to the patient's doctor office. Upon accompanying the patient to the doctor's office, the caregiver notices that his/her vehicle may be low on gas and cannot complete a round-trip back to the patient's house without any refilling the gas in his/her vehicle's tank. The next closest gas station may be two blocks away and beyond the approved permanent field of acceptability. In this situation, the caregiver would be given a prompt or alert in the user engagement panel to explain why he/she moved outside of the field of acceptability. It will be appreciated that establishing a temporary field of acceptability for this area outside of the field of acceptability, confirming or verifying that it is acceptable for the scope of services being provided, and then adjusting the field of acceptability to include the temporary field such that when a caregiver travels to the area again the system will recognize it as being within the field of acceptability and not issue an alert or notification. Reducing the frequency and/or number of alerts or notifications substantially improves the accuracy and efficiency of the billing verification system by reducing the data processing required. It will also be appreciated that establishing a reasonable length or distance the caregiver may travel off of an established route will also reduce the number of incoming prompts, alerts or notifications of this nature to the caregiver. Different shapes, sizes, and/or colors may be used to represent the permanent field of acceptability and the acceptable range. For example, the permanent field of acceptability may be represented by solid circle or other shape/region and colored green, whereas the acceptable range may be represented as a dotted circle extending beyond the green circle and colored yellow. All other unaccepted areas may be colored red.

A home health agency may track caregivers during their scheduled work times by using GPS within its tracking and communications device. Additionally, this GPS function will be able to reconcile both time and location problems simultaneously. This GPS function can provide the caregiver with additional prompts requiring constant interaction with the user engagement panel on the caregiver device. The main purpose is to ensure that the caregiver stays within the patient's home or other approved location and/or route and is providing care in accordance with the service request. For example, intervals will be set up throughout the day which requires both the patient and the caregiver to input information to ensure service is being provided. These intervals may be set for a customized number of minutes, hourly, and so forth. Additionally, the travel path of the visiting caregiver may be tracked and sent to the central server to ensure that the caregiver is not taking any detours unrelated to the course of employment.

In the situation of a billing error and/or a billing inquiry, where a payer attempts to verify the charges for a specific caregiver during a visit, it may be able to refer to the patient records with particularity. The system in the billing department may then query the field of acceptability tracking system to identify all events which occurred at a certain time. The system may then filter the data to identify only the relevant events being queried, such as whether the caregiver remembered to aid the patient in taking his/her medicine that day. Similarly, this applies to emergency situations where the agency may query the field of acceptability tracking system to ascertain whether a patient had any circumstances where a caregiver accompanied him/her to a hospital. The queries may be based off of the caregiver's start and stop times, the location of the patient, and/or the caregiver's location. Various service visit times and fields may be used to make this determination. All time and location information gathered by the system maybe be used to cross-reference other information, it is not necessarily limited to billing and emergency decisions.

The tracking performed by the GPS in the caregiver device may also separate locations of different patients located in close proximity to each other whom are patients under the same home healthcare company to avoid confusion. Since the GPS allows for real-time communication with the central server, it may geofence different fields of acceptability unique to each patient. The GPS component may also provide an indirect benefit for coordinators as a scheduling management system by tracking all of the caregivers who are providing service on-site, so it is apparent which caregivers are free to cover for other caregivers.

Contextual information may be used to update the field of acceptability creating an inference of the location of a caregiver and patient. Since the system has previously stored numerous time and location information associated with the pair, any form of action that may not be similar to the usual routine of time and location information may trigger an alert to the administrator or coordinator's system. An identification comparing what tasks were scheduled to be performed and what was actually being performed by the caregiver may be generated at this time. All or a portion of such information may be transmitted manually or automatically. A caregiver may prefer manual entry if they anticipate moving outside of the field of the acceptability during his/her shift and wish to update the system beforehand to avoid any future scheduling issues. The caregiver may be provided a list of common reasons in a drop-down menu or be given a fillable field in which to explain his/her reasons for the departure from the field of acceptability.

Verification between the caregiver and patient may be completed in a number of different ways. The tracking and communications device between the two may provide a method that is less susceptible to fraud than a simple signature. In certain embodiments, a facial recognition feature may be implemented into the communications device. A patient's signature may additionally be required to prove that the caregiver has arrived and performed all tasks. Voice, facial, retina, or other biometric recognition may also assist in the capture and storage of caregiver visit time and location information.

Many patients are often located within the same area. Thus, in certain embodiments, geo-fencing may be provided between patients. As such, within a populated area like New York City, many fields of acceptability may overlap one another, particularly if two patients live next door to each other, or within the same location on different floors, such as within an apartment building. In order to avoid the confusion of providing care to the wrong patient, each patient may be geo-fenced by the home health care administration. This geo-fencing may provide the necessary differentiation within in a closed location. Consequently, the fields of acceptability can also be made distinct from one another. This routine may be used to allow a home health care administrator to establish a virtual boundary around a predetermined patient location for purposes of automatically notifying the administrator when a caregiver crosses the boundary. The tracking and communications module must also have its own bi-lateral communication between the caregiver and the patient. To reduce the chance of fraud, the caregiver and patient may both be required to confirm the visit at simultaneous times. The tracking and communications module may be paired between caregiver and patient. In the case of, for example, husband and wife, or other cohabitants authorized as needing care by the health care agency, the tracking and communications module may also sync with those patients. Such synced modules may send all information collected to a central processing server located at the home health agency, which may further analyze and change the patient records to match the patient's needs. This central server may be used to determine different fields of acceptability based on data collected.

Patient privacy is of utmost importance as many laws govern how patient information may be disclosed. Certain measures may be implemented to maintain a patient's privacy. For example, each patient device in the tracking system may provide each patient with a patient ID. This ID may be used specifically for purposes of tracking a particular patient. Additionally, the caregiver may also be given their own caregiver ID for purposes of tracking that particular caregiver. The patient ID's in particular will limit all other patient information for any user roles other than the system administrator. The ID may be a combination of alphabetical letters and/or numbers that is unique to each patient and/or caregiver. In addition, a tracking system may be controlled to authenticate requesters for tracking information and may only give access to trusted requesters for tracking information or may provide access on an as-needed basis. For example, a requester may provide information to a tracking system regarding a particular procedure, and the tracking system may then obtain interface information about the procedure from another portion of a system to determine when the procedure occurred. A caregiver may also preset service limitations regarding the time(s) he/she is not available to provide service.

The field of acceptability may be previously established based on a past service request a caregiver completed or may be based on default parameters as mentioned above. The default parameters may depend on the area, such as within a large city, the time of day, the day of the week, etc. Also, the predetermined rules and the field of acceptability may be updated to respond to real-time circumstances. It is to be understood that “updating” the field of acceptability may be done by association, meaning that at least one of the underlying predetermined rules establishing the field of acceptability have been updated.

It will be appreciated that various embodiments of systems and methods disclosed herein provide automated caregiver tracking and home healthcare verification through computing devices, allow for greater communication between patients, caregivers, coordinators, and/or administrators through dynamically deploying relevant information through computing devices, and enable full service home healthcare services, including scheduling service requests, coordinating service of them, dynamically updating and facilitating changes to service requests, and providing real-time information to all parties during patient visits.

It will be understood that the phrases or terminology employed herein is for purposes of description and not limitation. While the inventive disclosure has been shown and described with reference to various preferred embodiments, those skilled in the art will readily appreciate that various changes and/or modifications may be made without departing from the spirit and scope of the inventive disclosure as defined by the claims. Any exemplary embodiments described herein are merely illustrative, and many variations can be introduced without departing from the spirit of the disclosure or from the scope of the appended claims. For example, elements and/or features of different exemplary embodiments may be combined with each other and/or substituted for each other. The scope of the inventive disclosure, therefore, shall be defined solely by the following claims, and it will be apparent to those of skill in the art that numerous changes may be made in such details without departing from the spirit and the principles of the inventive disclosure.

What is claimed is:

1. A computer-implemented method, comprising:
receiving a service request for a patient, the service request including a service location and a service time;
establishing a field of acceptability for complying with the service request in accordance with one or more predetermined rules;
periodically receiving location data and time data generated by a first computing device associated with a caregiver assigned to service the service request;
receiving a billing request associated with the service request;
automatically identifying, without user input, compliance with the service request by comparing the location data and the time data generated by the first computing device with the field of acceptability in accordance with the one or more predetermined rules; and
responsive to identifying compliance with the service request, automatically adjusting at least a portion of the location data generated by the first computing device to match the service location of the service request.

2. The method according to claim 1, wherein the field of acceptability includes the service location, a plurality of locations distinct from the service location, the service time, and a time period which includes the service time.

3. The method according to claim 1, wherein the first computing device generates the location data using a global positioning system (GPS) unit of a mobile device.

4. The method according to claim 1, further comprising:
transmitting, through a patient module, confirmation information from the patient confirming completion of the service request by the caregiver, wherein the information includes at least one of a signature, a PIN, a passcode, a fingerprint, or biometric data, and wherein the information includes at least a timestamp or location data.

5. The method according to claim 1, further comprising:
establishing a set of service rules defining the field of acceptability, the set of service rules including a predefined region for the service location, one or more locations not within the predefined region for the service location, the service time, and a time period which includes the service time; and
responsive to identifying compliance with the service request, modifying the set of service rules to include the location data generated by the first computing device in defining the field of acceptability.

6. The method according to claim 1, further comprising:
receiving service relevant data from one or more computing devices located at a distance from the service location;
aggregating the service relevant data in a database; and
updating the field of acceptability based on the aggregated service relevant data.

7. The method according to claim 6, wherein the aggregated service relevant data includes GPS data corresponding to one or more locations and time or time period corresponding to one or more times or dates of transmission of the aggregated service relevant data.

8. The method according to claim 1, wherein the automatically identifying compliance with the service request additionally comprises:
receiving, upon completion of the service request, confirmation from the patient affirming that the caregiver has completed the service request, and confirmation from the caregiver affirming that the caregiver has completed the service request, wherein at least one of the confirmation of the patient or the confirmation of the caregiver is submitted through a user engagement panel of the first computing device or a second computing device.

9. The method according to claim 1, wherein the service request includes an additional service location, and the one or more predetermined rules, in defining the field of acceptability, include the additional service location and a plurality of locations along a route between the service location and the additional service location.

10. A computer-implemented method, comprising:
receiving a service request for a patient, the service request including a service location and a service time;
establishing a field of acceptability in accordance with one or more predetermined rules for complying with the service request;
periodically receiving location data and time data generated by a first computing device associated with a caregiver assigned to service the service request;
automatically identifying, without user input, non-compliance with the service request by comparing the location data and the time data generated by the first computing device with the field of acceptability in accordance with the one or more predetermined rules; and
responsive to identifying the non-compliance with the service request, transmitting a notification to the caregiver of non-compliance with the service request, and at least one of:
(i) prompting the caregiver to comply with the service request; or
(ii) prompting the caregiver to submit at least one of a reason or evidence of a reason for the non-compliance with the service request.

11. The method according to claim 10, further comprising:
receiving, from the caregiver, service relevant data through a user engagement panel associated with the first computing device, wherein the service relevant data includes the reason or the evidence of the reason for the non-compliance, and wherein the service relevant data is in the form of at least one of text data, audio data, image data, or video data.

12. The method according to claim 11, further comprising:
receiving, from one or more additional caregivers through one or more computing devices, additional service relevant data, the one or more additional caregivers having firsthand experience with the patient or the service location; and
responsive to the service relevant data and the additional service relevant data, dynamically updating the field of acceptability.

13. The method according to claim 10, further comprising:
periodically receiving location data and time data generated by a second computing device associated with the patient; and
comparing at least one of:
(i) the field of acceptability with the location data and the time data generated by the second computing device; or
(ii) the location data and time data generated by the first computing device associated with the caregiver with the location data and time data generated by the second computing device associated with the patient to determine a relative distance between the caregiver and the patient during one or more time periods.

14. The method according to claim 13, further comprising:
responsive to both the first and second computing devices being outside of the field of acceptability but within a predetermined distance of one another:
(i) establish a temporary field of acceptability based on the location data and the time data of the first and second computing devices outside of the field of acceptability; and
(ii) receive an authorization from the patient affirming that the patient and the caregiver are in or travelling to one or more new locations approved by the patient in accordance with the service request or a new service request.

15. The method according to claim 14, further comprising:
responsive to receiving the authorization from the patient, incorporating the temporary field of acceptability into the field of acceptability.

16. The method according to claim 15, wherein incorporating the temporary field into the field of acceptability is contingent on receiving additional authorization from at least one of:
(i) the caregiver;
(ii) an agency which employs the caregiver; or
(iii) a third-party responsible for payment of claims submitted by the agency.

17. The method according to claim 15, wherein including the temporary field into the field of acceptability is contingent on the caregiver completing service outside the field of acceptability.

18. A computer-implemented system for verifying compliance with a home healthcare service, comprising:
a database for storing a plurality of service requests and first location data associated with a plurality of a patients, each service request including one or more requested services and an anticipated duration of the requested service;
a server configured to receive second location data generated by one or more location identifier units of computing devices associated with caregivers, wherein the second location data identifies locations of the computing devices determined using the location identifier units at times the caregivers are physically at the locations; and
one or more processors programmed to:
receive a service request for a patient, the service request including a service location and a service time;
establish a field of acceptability in accordance with one or more predetermined rules for complying with the service request;
periodically receive location data and time data generated by a first computing device associated with a caregiver assigned to service the service request;
receive a billing request associated with the service request;
automatically identify, without user input, compliance with the service request by comparing the service location and the service time, respectively, with the location data and the time data generated by the first computing device in accordance with the one or more predetermined rules; and
responsive to identifying compliance with the service request, automatically adjust at least a portion of the location data or the time data generated by the first computing device to match the service location or the service time of the service request.

19. The system according to claim 18, wherein the first computing device includes a camera configured for taking a photograph having metadata verifying use of the first computing device to take the photograph at a particular location and at a particular time.

20. The system according to claim 19, wherein the one or more processors are further configured to: responsive to not identifying compliance with the service request, transmit a notification to the caregiver identifying the non-compliance with the service request, and at least one of:
(i) prompt the caregiver to comply with the service request; or
(ii) prompt the caregiver to submit at least one of a reason or evidence of a reason for the non-compliance with the service request and enabling the caregiver to submit the photograph with the first computing device.
